(19) United States
(12) Patent Application Publication
(10) Pub. No.: US 2024/0238213 A1
(43) Pub. Date: Jul. 18, 2024

(54) IN VIVO ORAL INSULIN DELIVERY VIA COVALENT ORGANIC FRAMEWORKS

(71) Applicant: New York University in Abu Dhabi Corporation, Abu Dhabi (AE)

(72) Inventors: Ali TRABOLSI, Abu Dhabi (AE); Farah BENYETTOU, Abu Dhabi (AE)

(21) Appl. No.: 18/553,986

(22) PCT Filed: Apr. 5, 2022
(86) PCT No.: PCT/IB2022/000193

§ 371 Date: Oct. 4, 2023

Related U.S. Application Data
(60) Provisional application No. 63/170,967, filed on Apr. 5, 2021.

(51) Int. Cl. A61K 9/51; A61K 38/28; A61P 3/10; B82Y 5/00

(52) U.S. Cl. CPC A61K 9/5146; A61K 9/5192; A61K 38/28; A61P 3/10; B82Y 5/00

(57) ABSTRACT

Provided are imine-linked-covalent organic frameworks (nCOFs) nanoparticles. The COF nanoparticles may be formed from co-condensation of 2,6-diformylpyridine (DFP) and 4,4′,4″-(1,3,5-triazine-2,4,6-triyl)trianiline (TTA) monomers. The nanoparticles may be used to encapsulate cargo, such as insulin. The nanoparticles encapsulating insulin may be used to in a method to treat an individual having or suspected of having diabetes. The nanoparticles may be administered orally.

### CROSS REFERENCE TO RELATED APPLICATION

This application claims priority to U.S. Provisional Patent Application No. 63/170,967, filed on Apr. 5, 2021, the disclosure of which is incorporated herein by reference.

### BACKGROUND OF THE DISCLOSURE

As the seventh leading cause of death worldwide affecting nearly 10% of the world's population, a quadrupling of its prevalence since 1980 and accounting for almost 15% of direct healthcare costs, diabetes and its treatment is of global significance. Diabetes is a chronic disease occurring when no insulin is produced due the absence of pancreatic B-cell islets (type 1), or the insulin that is produced is incapable of being effectively utilized by the body (type 2). Coupled with lifestyle changes, insulin therapy remains a key element in controlling and regulating blood glucose levels, with the primary mechanism that of insulin injection. However, studies have shown delays in onset of insulin therapy by a large proportion of people with uncontrolled diabetes and with those who do eventually undertake treatment, there is a delay of more than 2 years. A fear of needles and self-injection as well as pain and anxiety are some of the many reasons why people are unwilling to start insulin therapy. Insulin pens alleviate some of these conditions, as well as overcome dosage issues that exist with vials and syringes, however this method is itself not error free. A shift towards oral delivery of insulin has the potential to improve the uptake of insulin therapy and revolutionize diabetes care since it is a noninvasive therapeutic approach without the side effects caused by frequent subcutaneous injection.

Orally delivered insulin is capable of reaching systemic circulation after passing through the liver similar to physiological insulin secretion, while subcutaneously injected insulin may result in peripheral hyperinsulinemia and associated complications. However, oral drug delivery faces numerous challenges including dissolution, bioavailability, solubility and its stability in the gastrointestinal (GI) tract. The oral bioavailability of insulin is severely hampered by its inherent instability in the GI tract and its low permeability across biological membranes in the intestine (less than 1%). Despite clinical trials of several oral insulin formulations, sufficient commercial development has not been yet achieved.

While nanocarriers such as polymeric, inorganic and solid-lipid nanoparticles have been used as insulin transporters, circumventing many of the problems associated with insulin oral delivery, recent clinical trials have resulted in failure due to toxicology, low levels of oral bioavailability and elevated intra-individual difference in insulin absorption; strong evidence that challenges still persist. Two systems have, so far, been FDA approved for the oral delivery of insulin. The first one, developed by Oramed (ORMD-0801) incorporates both a species-specific protease inhibitor that protects active ingredients, and a potent absorption enhancer that fosters their absorption across the intestinal epithelium. However the system is non-specific and its prolonged use may damage the stomach membrane barrier and may lead to toxicity. The second, HDV-I by Diasome is based on liposomes with hepatic targeting, which suffers from instability in the GIT, high cost and drug release during storage.

### SUMMARY OF THE PRESENT DISCLOSURE

In an aspect, the present disclosure provides imine-linked-covalent organic frameworks (nCOFs) nanoparticles. The COF nanoparticles may be formed from co-condensation of 2,6-diformylpyridine (DFP) and 4,4′,4″-(1,3,5-triazine-2,4,6-triyl)trianiline (TTA) monomers. For example, a COF nanoparticle may comprise a plurality of COF nanosheets of imine-linked 2,6-diformylpyridinyl monomers and 4,4′,4″-(1,3,5-triazine-2,4,6-triyl)trianilinyl monomers. In embodiments, the COF nanoparticle comprises 10-25 COF nanosheets (e.g., 10, 11, 12, 13, 14, 15, 16, 17, 18, 19, 20, 21, 22, 23, 24, or 25 COF nanosheets). In various examples, the COF nanoparticles have about 18 COF nanosheets.

In an aspect, the present disclosure provides methods of making COF nanoparticles. Also provided are methods of COF nanoparticles comprising one or more cargo molecules.

In an aspect, the present disclosure provides compositions. The composition may comprise COF nanoparticles incorporating cargo molecules (e.g., insulin).

In an aspect, the present disclosure provides methods of treating an individual having or suspected of having diabetes. The diabetes may be type 1 or type 2. In various examples, the individual has diabetes type 1.

In an aspect, the disclosure provides kits. A kit may comprise pharmaceutical preparations containing COF nanoparticles incorporated with insulin and printed material.

### BRIEF DESCRIPTION OF THE FIGURES

For a fuller understanding of the nature and objects of the disclosure, reference should be made to the following detailed description taken in conjunction with the accompanying figures.

FIG. 1. Insulin is intercalated between TTA-DFP-nCOF layers. a) Chemical structure and synthetic route of TTA-DFP-nCOF. b) HR-TEM image of TTA-DFP-nCOF. Cartoon representation illustrates the shape of TTA-DFP-nCOF. c) Structural model of TTA-DFP-nCOF, showing hcb layers that are disposed in abc sequence, generating hexagonal channels along the stacking direction. d) Schematic representation of the encapsulation of insulin between the layers of TTA-DFP-nCOF. Cartoon representation (spheres) illustrates the insulin. e) HR-TEM image of TTA-DFP-nCOF/Insulin. f) Confocal microscopy image of TTA-DFP-nCOF/Insulin-FITC; inset: fluorescence intensity. g) Van der Waals representation of the optimized location of insulin monomer molecule intercalated between TTA-DFP-nCOF layers. Atoms belonging to COF layers are displayed in white, gray, and black color, for each individual layer. For insulin molecule, C, N, O, and S atoms are shown. H atoms are omitted for clarity.

FIG. 2. TTA-DFP-nCOF/Insulin presents a glucose controlled release mode with delayed release and pH sensitivity. a) In vitro accumulated insulin-release from the TTA-DFP-nCOF/Insulin at 37° C. in PBS (10 mM) and pH 2.0, or pH 7.4 in several glucose concentrations ([glucose]=0, 1, 3, and 5 mg mL⁻¹). b) Pulsatile release profile of TTA-DFP-nCOF/Insulin-FITC at 37° C. as a function of glucose concentration ([glucose]=1 versus 5 mg mL⁻¹). Error bars indicate ±S.D. of triplicate experiments.

FIG. 3. TTA-DFP-nCOF/Insulin regulate glucose uptake in vivo without causing toxicity. a) blood glucose, b) serum insulin level changes and, c) oral glucose tolerance test (OGTT) versus time curves of the STZ-induced diabetic rats after oral administration of TTA-DFP-nCOF/Insulin at the insulin dosage of 50 IU·kg⁻¹. The group by subcutaneous injection (S.C.) of insulin at 5 IU·kg⁻¹ was set as a positive control. Glycemia, plasma insulin level and OGTT of diabetic rat (control, black) is also shown. Their corresponding area under the curve (AUC) is depicted in d), e) and f) for glycemia, plasma insulin levels and OGTT, respectively. TTA-DFP-nCOF/Insulin showed statistically significant differences in glycemia, plasma insulin levels and OGTT compared with S.C. insulin solution and diabetic control (*p<0.05; ** p<0.01; *** p<0.001). Each value represents mean #S.D. (n=3). g) Histopathological study. Sections of liver (i, ii, iii) and kidney (iv, v, vi) of control diabetic rat (I and iv), S.C. insulin (5 IU·kg⁻¹, ii and v) and TTA-DFP-nCOF/Insulin (50 IU·kg⁻¹, iii and vi) treated-diabetic rats. White arrow: big hepatocytes, red arrow: a necrosis of heatocytes and a narrowing in the sinusoids, *: Bowman capsules, grey arrow: glomeruli hypertrophy and tubules necrosis (black arrow).

FIG. 4. Synthetic route and chemical structure of TTA-DFP-nCOF.

FIG. 5. HR-TEM (a, b, c, d) and STEM (e, f) images of TTA-DFP-nCOF. Lattice fringe distances (d=0.4 nm) corresponding to the (110) plane of the nCOF and confirming the crystallinity of the material are also shown.

FIG. 6. Proposed mechanism of TTA-DFP-nCOF formation. Black arrows represent the stacking of the nanosheets due to the small presence of H₂O co-solvent which favors hydrogen bonding between nanosheets.

FIG. 7. HRTEM images of TTA-DFP-nCOF synthesized using 0.5 mL of acetic acid (17 M, [acetic acid]_{final}=5.0 M, no H₂O co-solvent).

FIG. 8. TEM image of TTA-DFP-nCOF suspended 24 hours at pH=2.0 showing no alteration of the nCOF structure.

FIG. 9. HR-TEM (a, b) and STEM (c, d) images of TTA-DFP-nCOF/insulin.

FIG. 10. HR-TEM (a, b) images and size distributions (c, d) of TTA-DFP-nCOF (a, c) and TTA-DFP-nCOF/insulin (b, d). In order to estimate the average size of the particles, an average of 300 particles were counted.

FIG. 11. Comparison of STEM images for TTA-DFP-nCOF (a, b) and TTA-DFP-nCOF/insulin (c, d).

FIG. 12. TEM mapping of sulfur element S in a) TTA-DFP-nCOF and b) TTA-DFP-nCOF/insulin. i) STEM image, ii) EDS mapping for S, iii) overlay of i) and ii) showing the localization of S elements in the nanoparticles. iv) Elemental analysis.

FIG. 13. AFM images (a, c) and height profiles (b, d) of TTA-DFP-nCOFs (a, b) and TTA-DFP-nCOFs/Insulin (c, d).

FIG. 14. Hydrodynamic diameter (a) and TEM images (b, c) of TTA-DFP-nCOF after synthesis (b) and after 12 months (c) in 100 mM HEPES buffer at pH 7.4. Inset: pictures of the solutions at t=0 and t=12 months. The experiment was performed in triplicate.

FIG. 15. PXRD patterns of pristine TTA-DFP-nCOF and TTA-DFP-nCOF/insulin (30% loading capacity) and TTA-DFP-nCOF/insulin (65% loading capacity).

FIG. 16. Nitrogen adsorption/desorption isotherms and pore size distribution curves (inset) at 77 K of TTA-DFP-nCOF before and after loading with Insulin. The experiment was performed in triplicate.

FIG. 17. Stacked FTIR spectra of TTA-DFP-nCOF and its precursors, 2,6-diformylpyridine (DFP) and 4,4′,4″-(1,3,5-triazine-2,4,6-triyl)trianiline (TTA). trz: triazine; ald: aldehyde; pyr: pyridine.

FIG. 18. Stacked FTIR spectra of insulin (top), TTA-DFP-nCOF (middle) and TTA-DFP-nCOF/insulin (bottom).

FIG. 19. Comparison of the FTIR spectra of insulin (top), TTA-DFP-nCOF (middle) and TTA-DFP-nCOF/insulin (bottom) between 1750-1350 cm⁻¹.

FIG. 20. a) TEM image, b) PXRD pattern and c) nitrogen adsorption/desorption isotherms of TTA-DFP-nCOF suspended for 24 hours at pH=2.0 showing no alteration of the nCOF structure.

FIG. 21. Monitoring insulin uptake into TTA-DFP-nCOF by 1H NMR at regular time intervals. Stacked ¹H NMR spectra of insulin in the absence of TTA-DFP-nCOF (pH adjusted to 7.4 with an initial concentration of insulin of 10 mM. bottom trace), and in the presence of TTA-DFP-nCOF at t=0, 30 min (minute), 1 h (hour), 1.5 h, 2 h, 3 h, 6 h, 9 h, 12 h, and 24 h in deuterated HEPES buffer solution in 500 MHz at 310 K.

FIG. 22. Calibration curve obtained by measuring the maximum fluorescence signal at different insulin-FITC concentrations (λₑₓ=488 nm, λₘₐₓ=520 nm, H₂O, 298 K).

FIG. 23. Fluorescence emission spectra of a) diluted supernatant solution of TTA-DFP-nCOF impregnated with insulin-FITC at t=0 hour and after 24 hours; b) TTA-DFP-nCOF, TTA-DFP-nCOF/insulin-FITC and Insulin-FITC. λₑₓ=488 nm, H₂O at pH 7.4, 298 K. The experiment was performed in triplicate.

FIG. 24. Confocal microscopy images of drop-cast TTA-DFP-nCOF/insulin-FITC on a cover slip to ensure immobilization of the NPs (λₑₓ=488 nm). The experiment was performed in triplicate.

FIG. 25. Hydrodynamic diameter (a) and TEM images (b, c) of TTA-DFP-nCOF/insulin after synthesis (b) and after 12 months (c) in 100 mM HEPES buffer at pH 7.4. The experiment was performed in triplicate.

FIG. 26. Zeta(ζ)-potential of insulin, TTA-DFP-nCOF, and TTA-DFP-nCOF/insulin at pH 7.4 in 100 mM HEPES. Error bars represent standard deviations of triplicate measurements.

FIG. 27. High resolution XPS spectra of TTA-DFP-nCOF. (a) XPS survey spectrum, and binding energy spectrum for (b) C1s, (c) O1s and (d) N 1s.

FIG. 28. High resolution XPS spectra of insulin. (a) XPS survey spectrum, and binding energy spectrum for (b) C1s, (c) O1s, (d) N 1s and (e) S 2s.

FIG. 29. High resolution XPS spectra of TTA-DFP-nCOF/insulin. (a) XPS survey spectrum, and binding energy spectrum for (b) C1s, (c) O1s, (d) N 1s and (e) S 2s.

FIG. 30. In vitro accumulated insulin-FITC release from the TTA-DFP-nCOF/insulin at 37° C. in human serum and a mix of amino acids for 24 hours. The % of drug released was measured using fluorescence emission. The experiment was performed in triplicate.

FIG. 31. In vitro accumulated insulin-FITC release from the TTA-DFP-nCOF/insulin at 37° C. in PBS containing a) fructose ([fructose]=3 mg·mL⁻¹) and b) sucrose ([sucrose]=3 mg·mL⁻¹) for 24 hours then glucose was added ([glucose]=3 mg·mL⁻¹) to trigger insulin-FITC release. The % of drug released was measured using fluorescence emission. The experiment was performed in triplicate.

FIG. 32. Circular dichroism spectra of native insulin solution and insulin incubated in gastro-intestinal environment. Deg=degree. The experiment was performed in triplicate.

FIG. 33. Circular dichroism spectra of native insulin solution and insulin released from the TTA-DFP-nCOF/insulin incubated in hyperglycemic conditions (5 mg·mL⁻¹) for 12 hours. Deg=degree. The experiment was performed in triplicate.

FIG. 34. Hydrodynamic diameter (a, c and e) and TEM images (b, d and f) of TTA-DFP-nCOF/insulin in 100 mM HEPES buffer at pH 7.4 (a, b), pH 2.0 (c, d) and in presence of lysozyme (5 mg·mL⁻¹, e, f) at t=0 hour and 24 hours. The experiment was performed in triplicate.

FIG. 35. Loading efficiency (wt %) of TTA-DFP-nCOF when incubated with insulin, glucose, and successively insulin followed by glucose at pH 7.4 in 100 mM HEPES.

FIG. 36. Zeta(ζ)-potential of TTA-DFP-nCOF, TTA-DFP-nCOF/insulin, TTA-DFP-nCOF/glucose and TTA-DFP-nCOF/insulin+glucose at pH 7.4 in 100 mM HEPES.

FIG. 37. TEM image a), PXRD pattern b), and nitrogen adsorption/desorption isotherms c), of TTA-DFP-nCOF loaded with glucose.

FIG. 38. TEM image a), PXRD pattern b), and nitrogen adsorption/desorption isotherms c), of TTA-DFP-nCOF/insulin after release in hyperglycemic conditions ([glucose]=5 mg·mL⁻¹).

FIG. 39. TEM images of a) TTA-DFP-nCOF, b) TTA-DFP-nCOF/insulin, and c) TTA-DFP-nCOF/insulin after release in hyperglycemic conditions.

FIG. 40. Viability of Hep-G2, HCT-116, HCT-8, RKO, HeLa, A2780, MDAMB-231, MCF-7, HEK-293 and U251-MG cells after 48 h incubation with TTA-DFP-nCOF or TTA-DFP-nCOF/insulin up to [TTA-DFP-nCOF]=1 mg·mL⁻¹. Error bars represent standard deviations of triplicate measurements.

FIG. 41. HCT-116 and RKO cells visualized by TEM (control cells).

FIG. 42. TEM images of HCT-116 cells treated with TTA-DFP-nCOF/insulin for 4 h at a) t=4 h, b) t=24 h, and c) t=48 h at various magnifications. White arrows show nanoparticle uptake in cells through endocytosis and their transit in the cytoplasm.

FIG. 43. TEM images of RKO cells treated with TTA-DFP-nCOF/insulin for 4 h at a) t=4 h, b) t=24 h, and c) t=48 h at various magnifications. White arrows show nanoparticle uptake in cells through endocytosis and their transit in the cytoplasm.

FIG. 44. Hemolysis activity of TTA-DFP-nCOF and TTA-DFP-nCOF/insulin. a) Photograph after centrifugation of fresh human blood incubated with different concentrations of TTA-DFP-nCOF and TTA-DFP-nCOF/insulin up to 3 mg·mL-I for 1 hour. b) Hemolysis rates (%) induced by different concentrations of TTA-DFP-nCOF and TTA-DFP-nCOF/insulin up to 3 mg·mL⁻¹. Physiological saline in the absence or the presence of Triton X-100 (0.3%) were respectively used as negative (C−) and positive (C+) controls. *** p<0.001, significantly different from negative control. Error bars represent standard deviations of triplicate measurements.

FIG. 45. Ex vivo permeation studies of TTA-DFP-nCOF/insulin-FITC. a) Apparent permeability profile of TTA-DFP-nCOF/insulin-FITC across mouse intestinal tissue in DMEM at 37° C. The formulations under study were syringed into intestinal sacs obtained from freshly excised mouse tissue. The filled tissues were incubated in oxygenated buffer at 37° C. Sample solution was withdrawn at fixed time intervals up to 180 min and replaced with fresh medium. Data are shown as the mean. Inset: intestinal sac containing 300 μL of TTA-DFP-nCOF/insulin-FITC (1 mg·mL⁻¹). Tests were carried out in triplicate on three different intestinal segments from three different mice. b) TEM micrograph of ex vivo intestinal tissue after 180 min of TTA-DFP-nCOF/insulin-FITC treatment showing the presence of TTA-DFP-nCOF/insulin (white arrows).

FIG. 46. TEM images (a, b) of the serosal medium of ex vivo permeation studies showing that TTA-DFP-nCOF can cross intact the intestinal barrier without change in morphology or size.

FIG. 47. TEM images of ex vivo intestinal tissues after 180 min of TTA-DFP-nCOF/insulin-FITC treatment showing the distribution of TTA-DFP-nCOF/insulin through the intestine (white arrows).

FIG. 48. TTA-DFP-nCOF/insulin regulate glucose uptake in vitro and in vivo. a) In vivo blood glucose level (of initial %) changes versus time curves of the STZ-induced diabetic rats after oral administration of TTA-DFP-nCOF/insulin and free-form insulin solution, all at an insulin dosage of 50 IU·kg⁻¹. The group by subcutaneous injection (S.C.) of insulin at 5 IU·kg⁻¹ was set as a positive control, while the group orally administrated with empty TTA-DFP-nCOF at 2 mg·kg⁻¹ served as a negative control. Blood glucose level of diabetic and non-diabetic rats are also shown. TTA-DFP-nCOF/insulin showed statistically significant differences in hypoglycemic effect compared with diabetic control (*p<0.05; ** p<0.01; *** p<0.001). Each value represents mean±S.D. (n=3).

FIG. 49. TTA-DFP-nCOF/insulin did not show toxicity in vivo. a) urea, b) creatinine levels and transaminase activities: c) aspartate aminotransferase (ASAT) and d) alanine aminotransferase (ALAT) activity of non-diabetic rats and the STZ-induced diabetic rats after oral administration of TTA-DFP-nCOF/insulin at the insulin dosage of 50 IU·kg⁻¹. The group by subcutaneous injection (S.C.) of insulin at 5 IU·kg-I was set as a positive control, while the group orally administrated with empty TTA-DFP-nCOF at 2 mg·kg⁻¹ served as a negative control. Diabetic rat levels (control) are also shown. (*p<0.05; ** p<0.01; *** p<0.001). Each value represents mean±S.D. (n=3).

FIG. 50. TTA-DFP-nCOF/Insulin regulate glucose uptake in vitro and in vivo. Glucose metabolism abilities of untreated HepG2 (HepG2), untreated resistant-HepG2 (R-HepG2), insulin-treated R-HepG2 (Insulin) and TTA-DFP-nCOF/Insulin 4 hours and 24 hours post-treatment. The values of HepG2 cells in the control experiments are defined as 100%. Each value represents mean±S.D. of triplicate experiments (*p<0.05; ** p<0.01; *** p<0.001).

FIG. 51. Viability of Hep-G2 cells after 48 h incubation with TTA-DFP-nCOF or TTA-DFP-nCOF/Insulin up to [TTA-DFP-nCOF]=1 mg·mL⁻¹. Error bars represent standard deviations of triplicate measurements.

FIG. 52. Hep-G2 cells visualized by TEM with a) no treatment (control), b) TTA-DFP-nCOF or c) and d) TTA-DFP-nCOF/Insulin for 4 hours. Arrows are showing the nanoparticles uptake in the cells through endocytosis, their transit in the cytoplasm, followed by their penetration in the nucleus via the nuclear pores and finally being excreted at the basolateral side of the cells. E=endocytose, M=mitochondria, N=nucleus.

FIG. 53. Confocal images of Hep-G2 cells incubated 4 hours with Insulin-FITC ([Insulin-FITC]=10 μM). Cells were observed using i) bright-field (BF) and ii) FITC channel (488 nm), and iii) overlay of BF and FITC channels. Scale bars=20 μm.

FIG. 54. Confocal images of Hep-G2 cells incubated for 4 hours with no additive (control) and a) a plasmic membrane marker, b) a lysosome marker, c) an actin marker, and d) a nucleus marker. i) FITC channel (λₑₓ=488 nm); ii) red channel (λₑₓ=647 nm) and iii) overlay of i) and ii). Scale bars=10 μm.

FIG. 55. Confocal images of Hep-G2 cells incubated for 4 hours with TTA-DFP-nCOF/Insulin-FITC ([Insulin-FITC]=10 μM) and a) a plasmic membrane marker, b) a lysosome marker, c) an actin marker, and d) a nucleus marker. i) FITC channel (λₑₓ=488 nm); ii) red channel (λₑₓ=647 nm) and iii) overlay of i) and ii). Scale bars=10 μm.

FIG. 56. Flow cytometry histograms quantifying Insulin-FITC fluorescence in Hep-G2 cells treated for 4 h with no additive (control), TTA-DFP-nCOF, Insulin-FITC, TTA-DFP-nCOF/Insulin-FITC, ([Insulin-FITC]=10 μM). Each histogram represents 50 000 cells.

FIG. 57. Viability of R-Hep-G2 cells after 48 h incubation with TTA-DFP-nCOF or TTA-DFP-nCOF/Insulin up to [TTA-DFP-nCOF]=1 mg·mL⁻¹. Error bars represent standard deviations of triplicate measurements.

### DETAILED DESCRIPTION OF THE DISCLOSURE

Although claimed subject matter will be described in terms of certain embodiments/examples, other embodiments/examples, including embodiments/examples that do not provide all of the benefits and features set forth herein, are also within the scope of this disclosure. Various structural, logical, and process step changes may be made without departing from the scope of the disclosure.

All ranges provided herein include all ranges and all values that fall within the ranges to the tenth decimal place, unless indicated otherwise.

In an aspect, the present disclosure provides imine-linked-covalent organic frameworks (nCOFs) nanoparticles. The COF nanoparticles may be formed from co-condensation of 2,6-diformylpyridine (DFP) and 4,4′,4″-(1,3,5-triazine-2,4,6-triyl)trianiline (TTA) monomers. For example, a COF nanoparticle may comprise a plurality of COF nanosheets of imine-linked 2,6-diformylpyridinyl monomers and 4,4′,4″-(1,3,5-triazine-2,4,6-triyl)trianilinyl monomers. In embodiments, the COF nanoparticle comprises 10-25 COF nanosheets (e.g., 10, 11, 12, 13, 14, 15, 16, 17, 18, 19, 20, 21, 22, 23, 24, or 25 COF nanosheets). In various examples, the COF nanoparticles have about 18 COF nanosheets.

Each nanosheet is formed from the co-condensation of 2,6-diformylpyridinyl 5 monomers and 4,4′,4″-(1,3,5-triazine-2,4,6-triyl)trianilinyl monomers. The monomers have the following structures (and are presented with their IUPAC names):

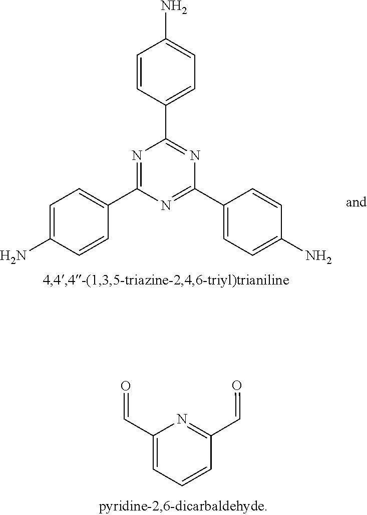 [SMILES: Nc1ccc(-c2nc(-c3ccc(N)cc3)nc(-c3ccc(N)cc3)n2)cc1 ; O=Cc1cccc(C=O)n1]

The nanosheets may have the following structure:

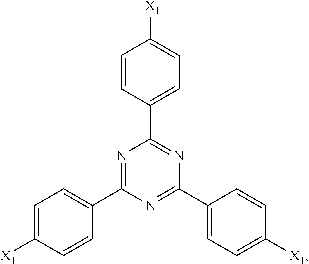

where X₁ is —NH₂,

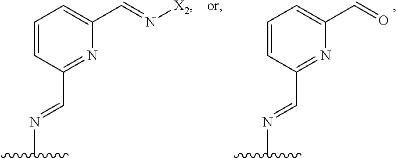

where X₂ is

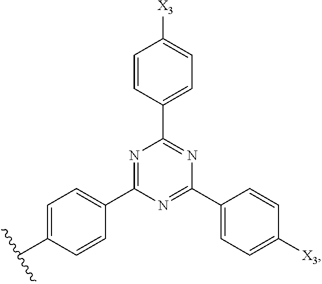

where X₃ is —NH₂,

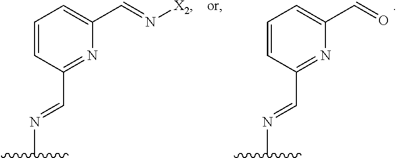

The nanosheets have a plurality of

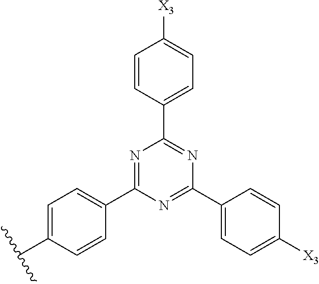

groups and/or

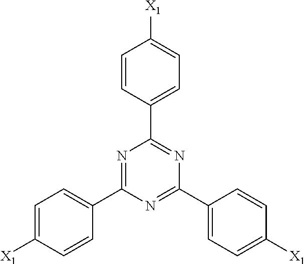

groups.

In various examples, at least one X₁ is not —NH₂.

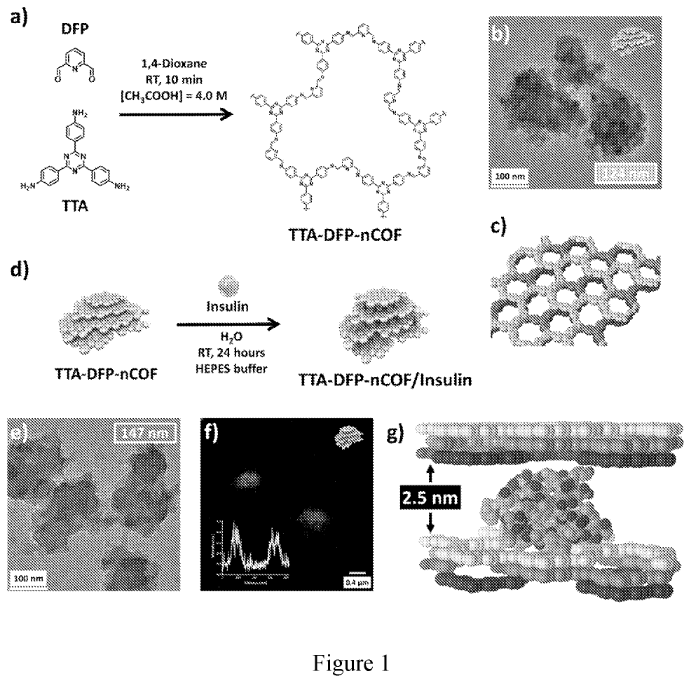

The layers may be stacked in a staggered configuration (see FIG. 1c). The COF nanosheets agglomerate such that a polycrystalline, spherical nanoparticle is formed. The COF network may have the following structure:

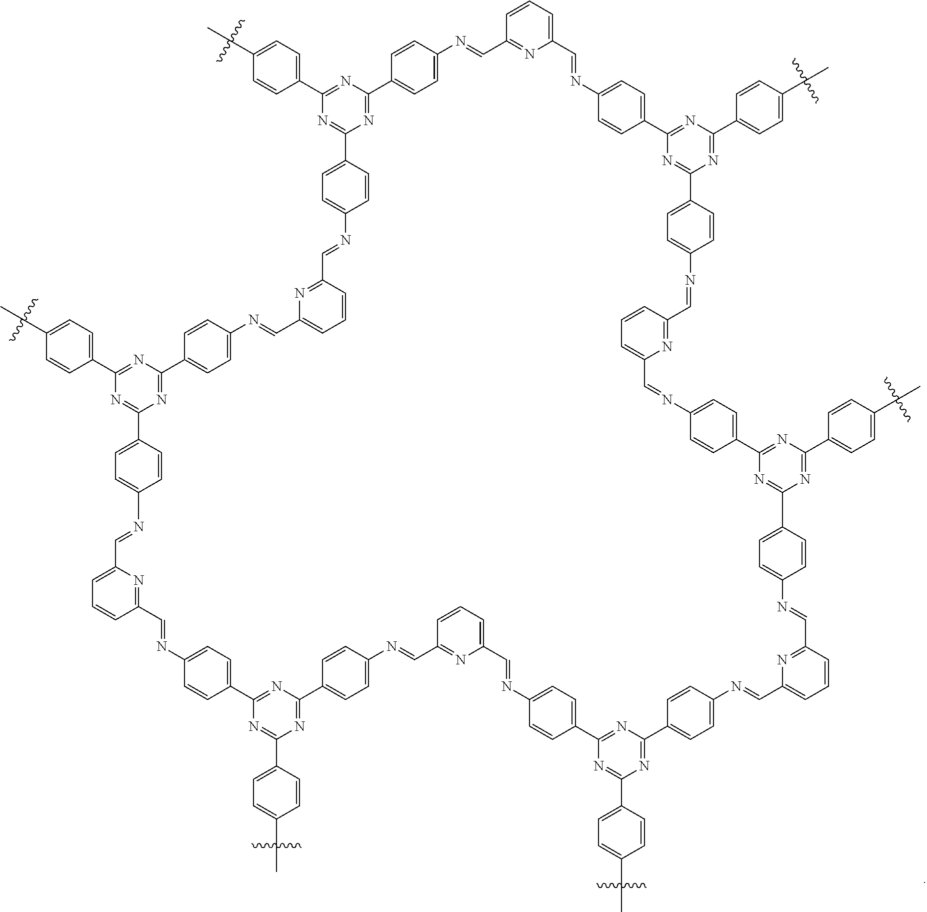 [SMILES: *c1ccc(-c2nc3nc(n2)-c2ccc(cc2)/N=C/c2cccc(n2)/C=N/c2ccc(cc2)-c2nc(-c4ccc(*)cc4)nc(n2)-c2ccc(cc2)/N=C/c2cccc(n2)/C=N/c2ccc(cc2)-c2nc(-c4ccc(*)cc4)nc(n2)-c2ccc(cc2)/N=C/c2cccc(n2)/C=N/c2ccc(cc2)-c2nc(-c4ccc(*)cc4)nc(n2)-c2ccc(cc2)/N=C/c2cccc(n2)/C=N/c2ccc(cc2)-c2nc(-c4ccc(*)cc4)nc(n2)-c2ccc(cc2)/N=C/c2cccc(n2)/C=N/c2ccc(cc2)-c2nc(-c4ccc(*)cc4)nc(n2)-c2ccc(cc2)N=Cc2cccc(n2)C=Nc2ccc-3cc2)cc1]

The layers may be hcb (honeycomb) layers disposed in an abc sequence. The staggered configuration may result in the formation of hexagonal channels along the stacking axis (see FIG. 1c). Cargo may be stored between layers. In various examples, the layers are crystalline and porous.

The COF nanoparticles may have various sizes. For example, the COF nanoparticles have a longest linear dimension (e.g., diameter) of 10-300 nm, including every 0.1 nm value and range therebetween (e.g., 75-300 nm). In various examples, the COF nanoparticles have a longest linear dimension (e.g., diameter) of 100-150 nm. In various other examples, the COF nanoparticles have a longest linear dimension (e.g., diameter) of about 120 nm.

The COF nanoparticles may be used as carriers for delivery of cargo molecules, such as proteins.

In embodiments, the present nanoparticles may be used for delivery of insulin. For example, insulin loaded COF nanoparticles may be used for glucose-responsive oral insulin delivery to overcome insulin oral delivery barriers. The gastro-resistant nCOF was prepared of layered nanosheets with insulin loaded between the nanosheets. The insulin-loaded nCOF exhibited insulin protection in digestive fluids as well as a glucose-responsive release.

The unique features of this delivery method are its desirable insulin-loading capacity (˜65 wt %), biocompatibility, insulin protection under harsh conditions and a hyperglycemic-induced drug release. Insulin-loaded TTA-DFP-nCOF successfully crossed the intestinal barrier and sustainably reduced the blood glucose level in vivo on diabetic rats (TID) with complete return to normal glucose level as compared to the non-diabetic rat control group without inducing systemic toxicity. The COF nanoparticles offer better storage and physiological stability compared to other nanosized colloidal carriers such as liposomes and emulsions, with nanoscale imine-linked covalent organic frameworks (nCOFs) in particular having shown tremendous potential as emerging nanomedicine candidates for drug delivery. nCOFs also feature a long-range ordered structure in which the organic building blocks are spatially controlled in two or three dimensions leading to regular pores with diameters facilitating the loading and controlled release of large drugs and proteins/enzymes. In addition, their high flexibility in molecular architecture and functional design make them versatile and therefore give them unique responsivity to their environment.

These nCOFs transports insulin molecules into insulin-resistant cells, demonstrating their potential in glucose upregulation and the disappearance of insulin resistance symptoms to treat type 2 diabetes. This is a strong evidence that nCOF based insulin oral delivery systems could replace traditional subcutaneous injections easing insulin therapy.

In comparison to the two FDA-approved technologies, the system described herein is biocompatible, highly stable in the stomach, cost effective, specific and glucose-responsive, therefore represent a step forward in the future of insulin oral delivery and a pathway for the treatment of type 1 and 2 diabetes through nCOF-based insulin oral delivery.

The COF nanoparticles may incorporate various types of cargo molecules. For example, the COF nanoparticles incorporate proteins. In various examples, the protein is insulin. The insulin molecules may be incorporated between the COF layers (see FIGS. 1(d) and 1(g)). For example, the concentration of incorporated insulin is 10-75 wt %, including every 0.1 wt % value and range therebetween, relative to the weight of the nanoparticle. The weight percent of incorporated insulin may reflect a ratio of insulin to the nanoparticle. In various examples, the concentration of incorporated insulin is 30-75 wt %. In various other examples, the concentration of incorporated insulin is about 65 wt %.

The COF nanoparticles may release its cargo upon exposure to an external trigger. Although the COF nanoparticles may exhibit a slower, natural, release of protein cargo, exposure to an external trigger may increase the release of protein cargo. For example, the COF nanoparticles may release incorporated insulin at a faster rate upon exposure to glucose.

The COF nanoparticles may have one or more desirable features. For example, the nanoparticles have a desirable incorporating capacity, are not damaged in acidic environments (e.g., the environment of the stomach), and a glucose-responsive release.

In an aspect, the present disclosure provides methods of making COF nanoparticles. Also provided are methods of COF nanoparticles comprising one or more cargo molecules.

In an example, a method for making covalent organic framework nanoparticles of the present disclosure comprises contacting 2,6-diformylpyridine (DFP) and 4,4′,4″-(1,3,5-triazine-2,4,6-triyl)trianiline (TTA) in a solvent and an acid to form a reaction mixture, wherein the reaction mixture is held at room temperature. The reaction mixture is maintained at room temperature for 1-30 minutes (e.g., 10 minutes). The DFP and TTA co-condense to form imine linked COF networks. The COF layers stack forming polycrystalline, spherical, porous nanoparticles. Various organic solvents may be used, such as, for example, 1,4-dioxane. Various acids may be used, such as, for example, acetic acid.

A method of the present disclosure further comprises purifying reaction mixture comprising the COF nanoparticles. For example, the purifying comprises dialyzing the reaction mixture in water. The dialysis may be performed for various lengths of time.

Various ratios of DFP to TTA may be used. For example, the molar ratio of DFP to TTA is 5:1.

A method may further comprise loading the COF nanoparticles with one or more cargo molecules. Examples of cargo molecules are provided herein. The cargo molecule may be a cargo protein. For example, the cargo molecule is insulin. The loading may be achieved by an impregnation method. For example, the loading comprising forming a reaction mixture of the COF nanoparticles and insulin. The reaction mixture may be buffered with, for example, HEPES buffer (pH 7.4). The reaction mixture may be allowed to stir for a length of time (e.g., 24 hours) at room temperature. The weight ratio of COF nanoparticles to insulin may be 1:2 to 2:1, including every 0.1 ratio value and range therebetween. Loading may further comprise one or more additional purification steps. Purification may comprise centrifugation and rinsing with water.

In an aspect, the present disclosure provides compositions. The composition may comprise COF nanoparticles incorporating cargo molecules (e.g., insulin).

The composition can comprise COF nanoparticles incorporating insulin in a pharmaceutically acceptable carrier (e.g., carrier). The carrier can be an aqueous carrier suitable for administration to individuals including humans. The carrier can be sterile. The carrier can be a physiological buffer. Examples of suitable carriers include sucrose, dextrose, saline, and/or a pH buffering element (such as, a buffering element that buffers to, for example, a pH from pH 5 to 9, from pH 6 to 8, (e.g., 6.5)) such as histidine, citrate, or phosphate. Additionally, pharmaceutically acceptable carriers may be determined in part by the particular composition being administered. Accordingly, there are a wide variety of suitable formulations of pharmaceutical compositions of the present disclosure. Additional, non-limiting examples of carriers include solutions, suspensions, and emulsions that are dissolved or suspended in a solvent before use, and the like. The composition may comprise one or more diluents. Examples of diluents, include, but are not limited to distilled water, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2012) 22nd Edition, Philadelphia, PA. Lippincott Williams & Wilkins. For example, a composition comprises a plurality of COF nanoparticles incorporating insulin, and a sterile, suitable carrier for administration to individuals including humans—such as a physiological buffer such as sucrose, dextrose, saline, pH buffering (such as from pH 5 to 9, from pH 7 to 8, from pH 7.2 to 7.6, (e.g., 7.4)) element such as, for example, histidine, citrate, or phosphate. In various examples, the composition may be suitable for injection. Parenteral administration includes infusions and injections, such as, for example, intramuscular, intravenous, intraarterial, intraperitoneal, subcutaneous administration, and the like.

In an aspect, the present disclosure provides methods of treating an individual having or suspected of having diabetes. The diabetes may be type 1 or type 2. In various examples, the individual has diabetes type 1.

A method for treating an individual having or suspected of having diabetes may comprise orally administering to the individual COF nanoparticles incorporating insulin. The COF nanoparticles incorporating insulin may be administered as a composition comprising a pharmaceutically acceptable carrier. Following oral administration, the individual's glucose levels are normalized. Normalized glucose levels are known in the art as normal fasting blood glucose levels of an individual whom is not diabetic. For example, expected values for normal fasting blood glucose concentration are in the range of 70 mg/dL to 100 mg/dL.

For oral administration, various formulations for the composition comprising the COF nanoparticles incorporating insulin may be used. Illustrative examples of formulations include, but are not limited to, gels, pills, tablets, solutions, and the like.

In various examples, the method comprises administering the composition or COF nanoparticles via non-oral routes, such as, for example, injection.

In an aspect, the disclosure provides kits. A kit may comprise pharmaceutical preparations containing COF nanoparticles incorporated with insulin and printed material.

In various examples, a kit comprises a closed or sealed package that contains the pharmaceutical preparation. In various examples, the package comprises one or more closed or sealed vials, bottles, blister (bubble) packs, or any other suitable packaging for the sale, or distribution, or use of the compounds and compositions comprising compounds of the present disclosure. The printed material may include printed information. The printed information may be provided on a label, or on a paper insert, or printed on the packaging material itself. The printed information may include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material may include an indication that the pharmaceutical composition and/or any other agent provided with it is for treatment of a subject having diabetes type 1 or diabetes type 2. In various examples, the product includes a label describing the contents of the container and providing indications and/or instructions regarding use of the contents of the container to treat a subject having diabetes type 1 or diabetes type 2. A kit may comprise a single dose or multiple doses.

The following Statements provide examples of the present disclosure.
Statement 1. A covalent organic framework (COF) nanoparticle, comprising 10-25 COF nanosheets, wherein the COF nanosheets are stacked in a staggered configuration and each COF nanosheet is a co-condensate of 2,6-diformylpyridine (DFP) and 4,4′,4″-(1,3,5-triazine-2,4,6-triyl)trianiline (TTA) and the COF nanoparticle has a longest linear dimension of 75-300 nm.
Statement 2. A COF nanoparticles according to Statement 1, wherein the molar ratio during condensation of DFP and TTA is 5:1 (DFP:TTA).
Statement 3. A COF nanoparticle according to Statements 1 or 2, wherein the COF nanoparticle has a longest linear dimension of about 120 nm.
Statement 4. A COF nanoparticle according to any one of the preceding Statements, wherein the COF nanoparticle has 16-20 COF nanosheets.
Statement 5. A COF nanoparticle according to any one of the preceding Statements, wherein the COF nanoparticle has 18 COF nanosheets.
Statement 6. A COF nanoparticle according to any one of the preceding Statements, wherein the COF nanoparticle incorporates a plurality of cargo proteins.
Statement 7. A COF nanoparticle according to Statement 6, wherein the cargo protein is insulin.
Statement 8. A composition comprising a plurality of COF nanoparticles according to any one of the preceding Statements and a pharmaceutically acceptable carrier.
Statement 9. A composition according to Statement 8, wherein the composition is suitable for oral consumption.
Statement 10. A method for making covalent organic framework (COF) nanoparticles, comprising: contacting 2,6-diformylpyridine (DFP), 4,4′,4″-(1,3,5-triazine-2,4,6-triyl)trianiline (TTA), and an acid in a solvent to form a reaction mixture, wherein the reaction mixture is held at room temperature, wherein after a period of time (e.g., 10 minutes), the covalent organic framework nanoparticles are formed.
Statement 11. A method according to Statement 10, further comprising purifying the reaction mixture, wherein the purifying comprises dialyzing the reaction mixture in water.
Statement 12. A method according to Statement 10 or Statement 11, wherein the COF nanoparticles further comprise one or more cargo proteins.
Statement 13. A method according to Statement 12, wherein the one or more cargo proteins are insulin.
Statement 14. A method according to Statement 12 or Statement 13, wherein the one or more cargo proteins are loaded via impregnation.
Statement 15. A method according to Statement 14, wherein impregnation comprises: forming a reaction mixture comprising the COF nanoparticles and insulin at room temperature, wherein the COF nanoparticles are impregnated with one or more insulin molecules.
Statement 16. A method according to Statement 15, wherein the reaction mixture is a buffered aqueous mixture.
Statement 17. A method according to Statement 16, wherein the reaction mixture is buffered with HEPES at a pH of 6 to 8 (e.g., 7.4).
Statement 18. A method according to any one of Statements 15-17, further comprising purifying the impregnated COF nanoparticles.
Statement 19. A method according to Statement 18, wherein the purifying comprises centrifugation and washing with water.
Statement 20. A method according to any one of Statements 10-19, wherein the COF nanoparticles have a longest linear dimension of 75-300 nm (e.g., 120 nm).
Statement 21. A method according to any one of claims 10-20, wherein the COF nanoparticles have 10-25 COF layers (e.g., 18 COF layers).
Statement 22. A method according to any one of Statements 10-21, wherein the molar ratio of DFP to TTA is 5:1.
Statement 23. A method according to any one of Statements 10-22, wherein the solvent is 1,4-dioxane.
Statement 24. A method according to any one of Statements 10-23, wherein the acid is acetic acid.
Statement 25. A method according to any one of Statements 14-24, wherein the weight ratio of COF nanoparticles to insulin is 1:2 to 2:1.
Statement 26. A method for treating an individual having or suspected of having diabetes, comprising orally administering a composition of Statements 8 or 9, wherein the individual's glucose levels are normalized.
Statement 27. A method according to Statement 26, wherein the individual has diabetes type 1 or diabetes type 2.
Statement 28. A method according to Statement 27, wherein the individual has diabetes type 1.
Statement 29. A kit comprising COF nanoparticles according to any one of Statements 1-7 or a composition or components to prepare a composition according to Statement 8 or Statement 9.

The following examples are presented to illustrate the present disclosure. They are not intended to be limiting in any matter.

### Example 1

This example provides a description of COF nanoparticles and COF nanoparticles incorporating insulin and methods of making and using same.

Herein, imine-based nCOF obtained from the co-condensation of 2,6-diformylpyridine (DFP) and 4,4′,4″-(1,3,5-triazine-2,4,6-triyl)trianiline (TTA) (noted TTA-DFP-nCOF), was prepared as highly crystalline nanoparticles using a seeded growth methodology and was successfully used as an oral insulin delivery system. The choice of the triazine-based TTA-DFP-nCOF was primarily based on its high stability under harsh conditions including acidic environments. The unique features of this delivery method are its high insulin-loading capacity (˜65 wt %), biocompatibility, insulin protection under harsh conditions and a hyperglycemic-induced drug release. Insulin-loaded TTA-DFP-nCOF succesfully crossed the intestinal barrier and sustainably reduced the blood glucose level in vivo on diabetic rats (TID) with complete return to normal glucose level as compared to the non-diabetic rat control group without inducing systemic toxicity. In comparison to the two FDA-approved technologies, this system is biocompatible, highly stable in the stomach, cost effective, specific and glucose-responsive, therefore represents a step forward in the future of insulin oral delivery and a novel pathway toward the treatment of type 1 and 2 diabetes through nCOF-based insulin oral delivery.

### Results and Discussion

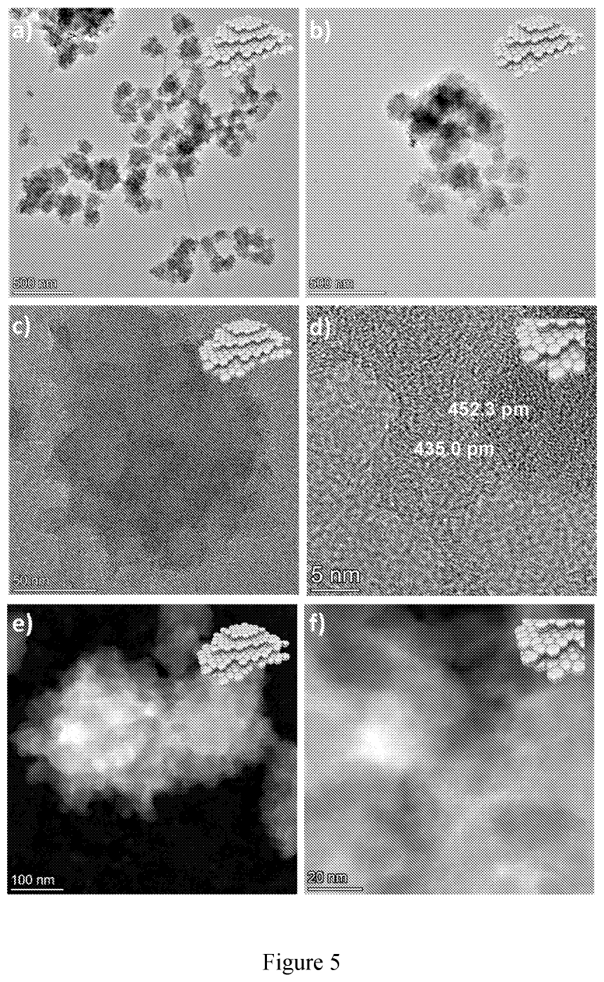

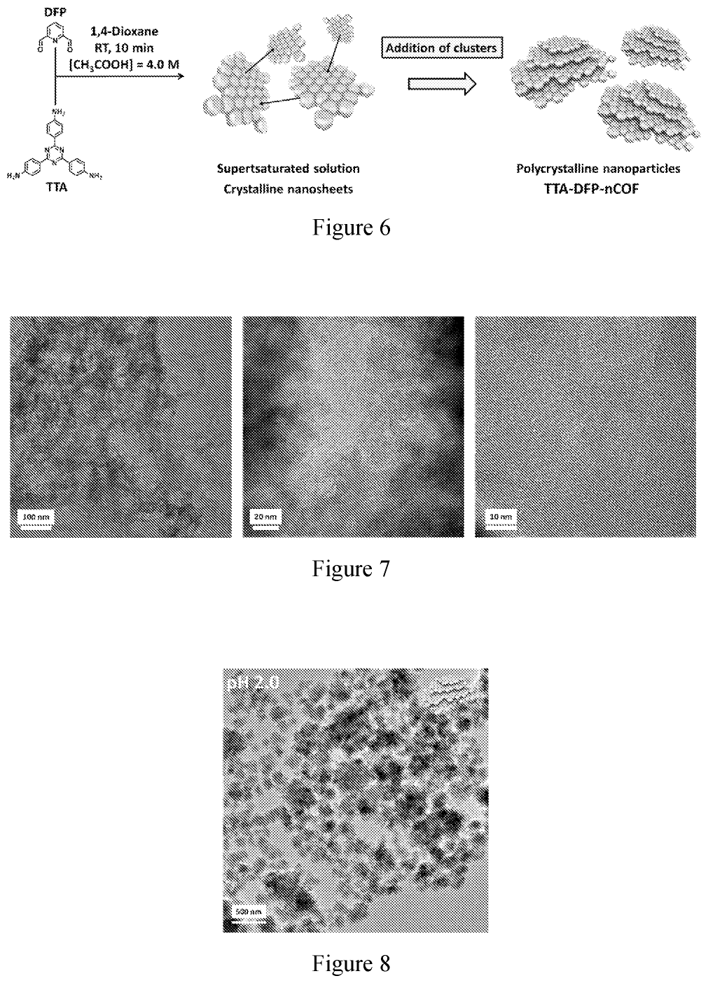

TTA-DFP-nCOF was synthesized by co-condensation of DFP (21 mg, 0.15 mmol, 5 equivalents) and TTA (12 mg, 0.03 mmol, 1 equivalent), in anhydrous 1,4-dioxane (3 mL) in the presence of acetic acid (0.5 mL, 13 M, [acetic acid]_{final}=4.0 M) at room temperature for 10 min (FIG. 1a). The solution was cleaned using dialysis in H₂O to obtain a stable nanoparticle suspension. At room temperature for 10 minutes, imine-linked covalent organic nanoparticles with 123.7 nm average diameter (FIGS. 1b and 5) emerge from clear solution without forming amorphous polyimine precipitates. A high concentration of acetic acid ([acetic acid]_{final}=4.0 M) induces a rapid imine condensation reaction at room temperature and thus the formation of discrete nCOF crystalline nanosheets (FIGS. 5-6). The increased rate of monomer consumption induces both supersaturation in crystalline nanosheets and inhibition of crystallite growth into bigger structures. Subsequently, nanosheets agglomerate by stacking to each other to form polycrystalline nanoparticles of spherical shape with rough surfaces and small protrusions (FIG. 6); this latter phenomenon is due to the small presence of H₂O co-solvent which favors hydrogen bonding between nanosheets. When the synthesis is performed with pure acetic acid in the absence of H₂O ([acetic acid]_{final}=5.0 M), small crystalline nanosheets with limited stacking were obtained without observing nanoparticle formation (FIG. 7).

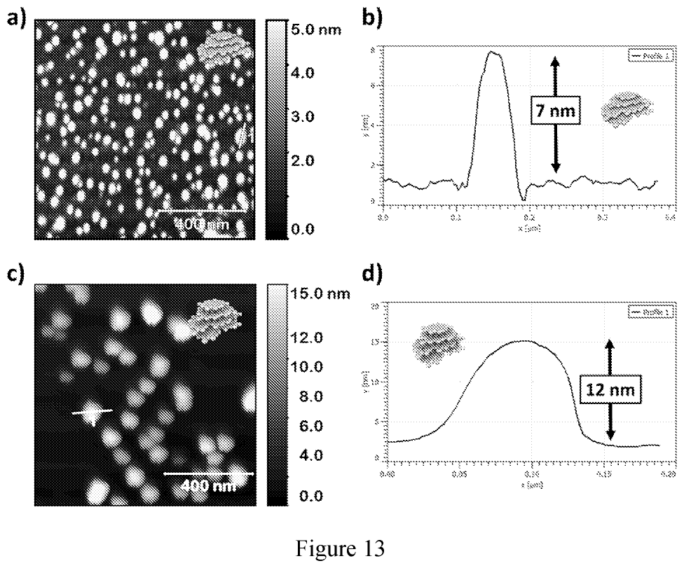

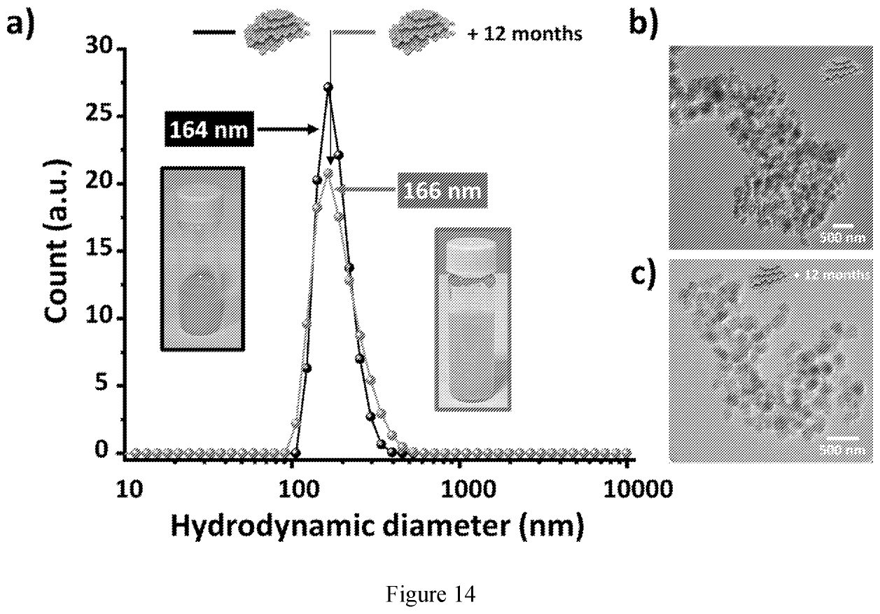

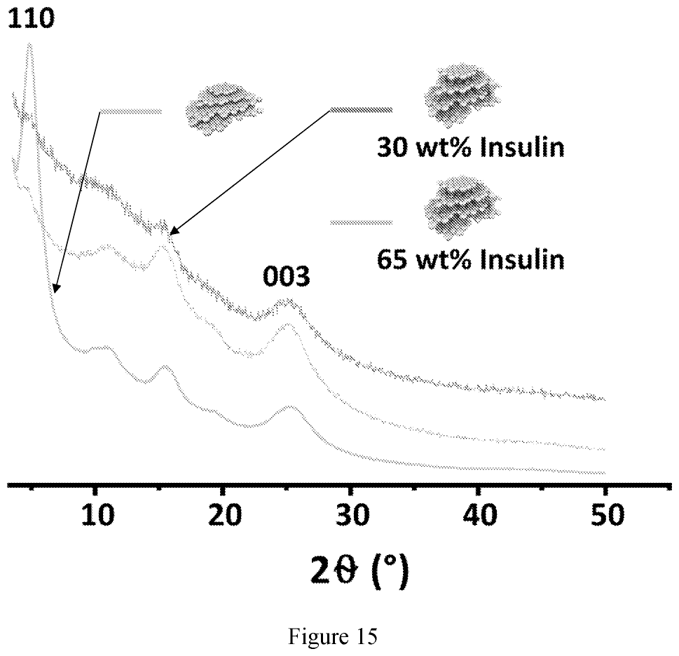

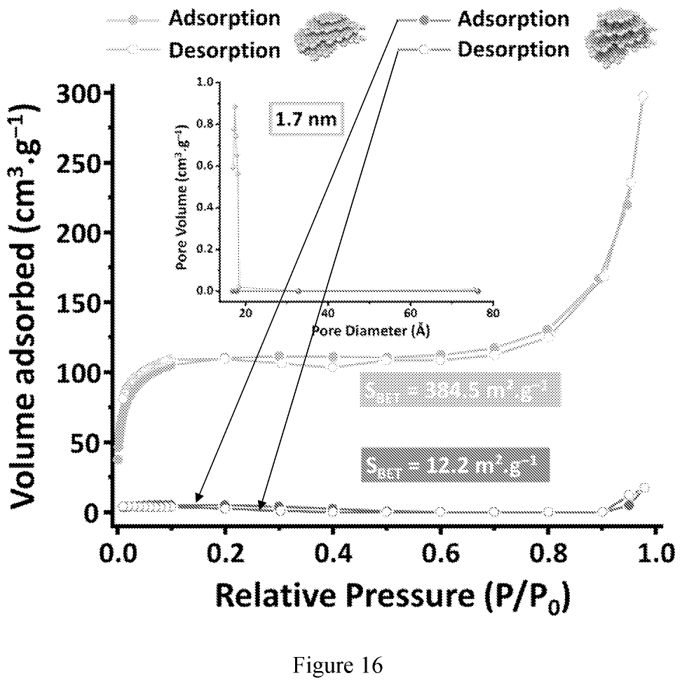

From a drop-cast solution (FIG. 13), under atomic force microscopy (AFM) TTA-DFP-nCOF appears as uniform particles with a height of 7 nm, corresponding to the stacking of ˜ 18 nCOF layers. In solution, they present themselves as particles with an average hydrodynamic radius of 68 nm with a polydispersity (PDI) of 0.1 from dynamic light scattering (DLS) analysis, with no precipitation observed over time. Once isolated as solids using a freeze-drying process, the nanoparticles could be re-dispersed in H₂O and were stable in solution for at least twelve months, during which their size distribution remained unchanged (FIG. 14). Crystallinity of the as-synthesized material was demonstrated by powder X-ray diffraction (PXRD), which features a strong peak at 2θ=4.9° assigned to the (110) plane of the regularly ordered lattice, as well as a broad peak centered at 2θ=25.6°, corresponding to the reflection from the (003) plane (FIGS. 1c and 15). Seed-mediated crystallization can explain the increased crystallinity as the amorphous imine polymer formation commonly observed in nCOF powders is not observed. N₂ sorption demonstrates the permanent porosity of nCOF. The shape of the isotherm combines type I and II features, and the Brunauer-Emmett-Teller (BET) surface area is 384.5 m² g⁻¹ (FIG. 16). The isotherm displays a H3-type hysteresis indicative of aggregates of plate-like particles giving rise to slit-shaped pores, similar to those of nanosheets-based materials reported elsewhere.

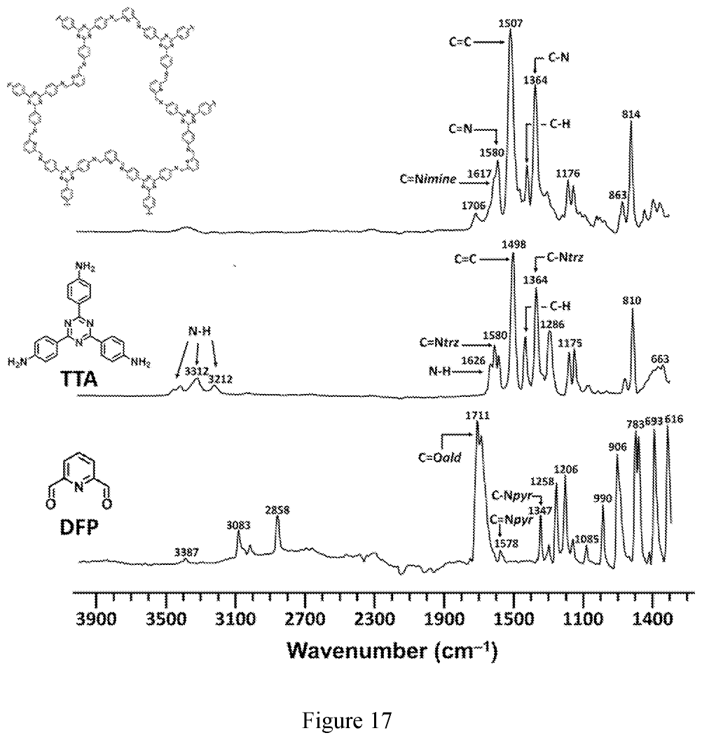

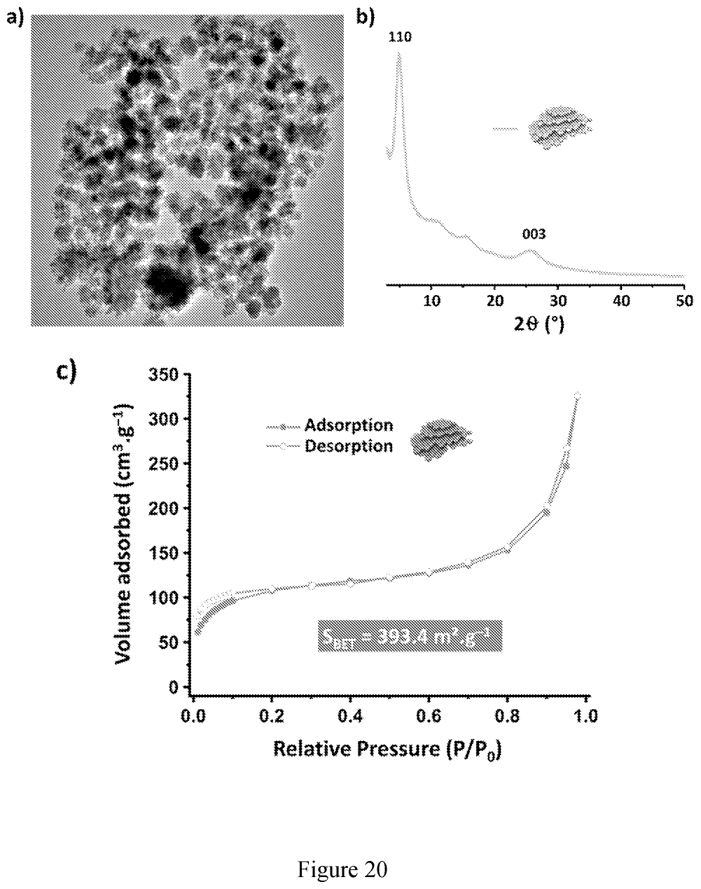

FTIR analysis of the nCOF shows the characteristic imine stretch at 1617 cm⁻¹, a peak at 1582 cm⁻¹ attributed to the C—C═N of the triazine and pyridine stretch, a broad and a strong peak at 1365 cm⁻¹ corresponding to the C—N of the triazine core and a strong vibration peak at 1507 cm⁻¹ corresponding to the C═C aromatic bonds along with the disappearance of the TTA primary amine N—H stretch at ˜3320 cm 1 and the DFP aldehyde stretch at 1711 cm⁻¹ (FIG. 17). The chemical stability of the TTA-DFP-nCOF in conditions designed to simulate the stomach environment (pH 2.0) as well as the bloodstream (pH 7.4) was assessed. TTA-DFP-nCOF remained unaffected in both conditions as demonstrated through TEM imaging (FIGS. 5-8), PXRD and BET (FIG. 20). Because of these desirable features, the COF nanoparticles were used for insulin oral delivery. Collectively, these bulk characterizations indicated that the nanosized TTA-DFP-nCOF nanoparticles are of high quality imine-linked nCOFs.

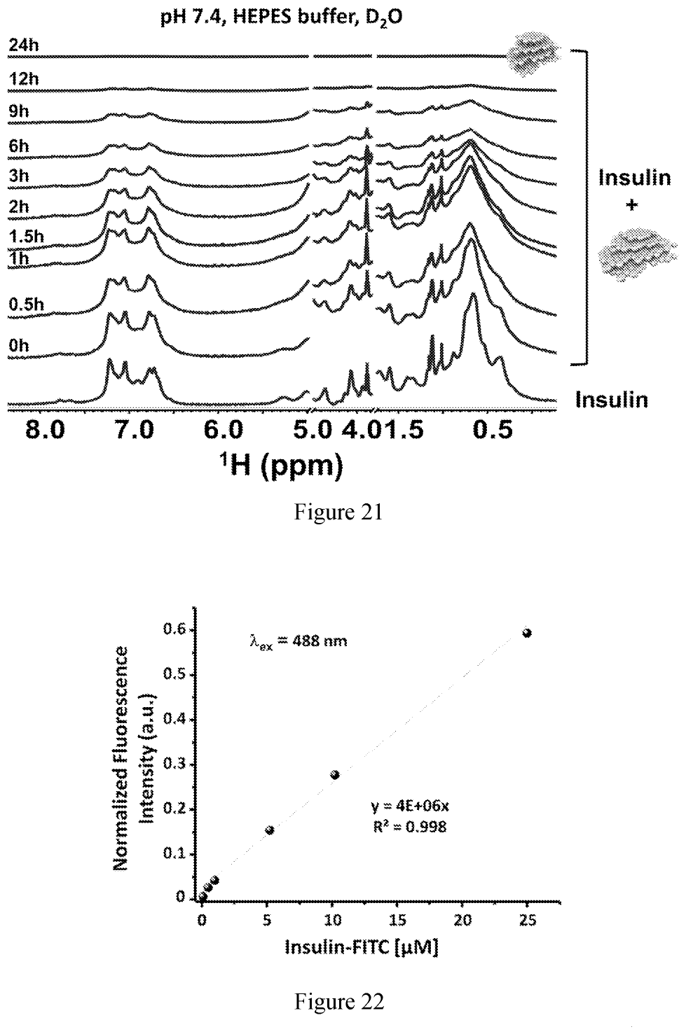

Insulin-loading of TTA-DFP-nCOF was first realized by soaking TTA-DFP-nCOF (5 mg) in insulin solution (5 mg in 2.5 mL HEPES buffer, pH 7.4) and monitored using 1H NMR over a period of 24 hours (FIG. 21). The signal of the insulin disappeared progressively once the nanoparticles are added to reach complete disappearance after 24 hours due to their encapsulation inside the TTA-DFP-nCOF.

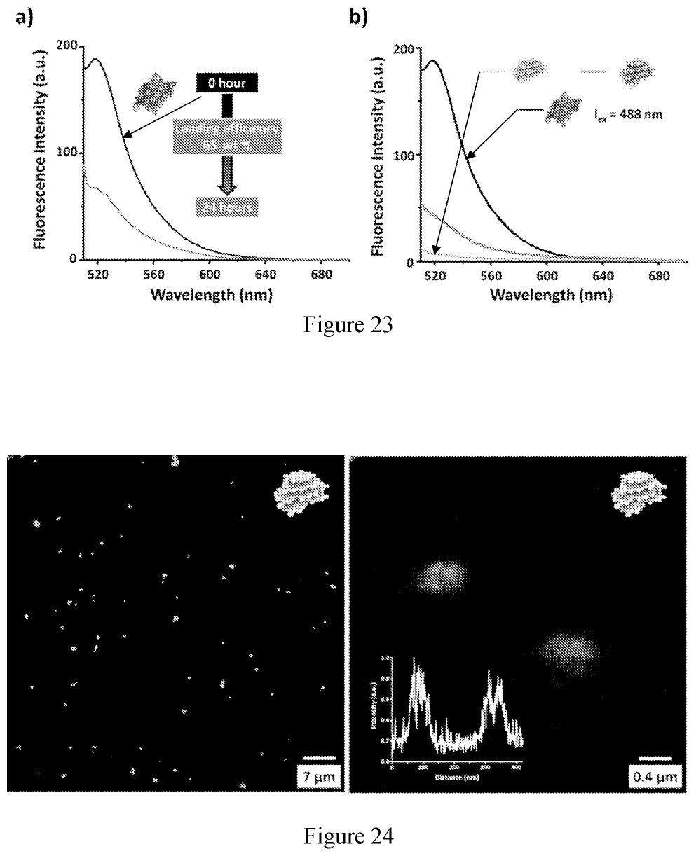

The use of dye (FITC)-labelled-insulin (insulin-FITC) facilitates the quantification of the amount loaded insulin. Insulin-FITC uptake was measured using fluorescence spectroscopy of the supernatant to quantify the concentration of unloaded insulin-FITC (FIGS. 22-23) with TTA-DFP-nCOF exhibiting an insulin-FITC loading capacity of 64.6±1.7 wt % (FIG. 23), comparable to previously reported insulin encapsulation in porous materials (Table 4). In order to visually show the presence of the insulin-FITC inside the NPs, confocal microscopic investigation was performed. Fluorescent nature of the FITC-insulin loaded TTA-DFP-nCOF (λ=488 nm, FIGS. 1f and 24) confirmed the presence of FITC-insulin in the nanomaterial.

Upon insulin loading, PXRD patterns of TTA-DFP-nCOF/Insulin was nearly flat, as compared to the pristine TTA-DFP-nCOF (FIG. 15, Table 1). TTA-DFP-nCOF was loaded with a lesser amount of insulin (30% loading capacity), and the PXRD pattern shows the presence of 2θ=4.9° and 25.6° peaks, although their intensities have decreased significantly compared to the pristine nCOF. It is anticipated that upon loading with insulin, the periodicity in the TTA-DFP-nCOF layers is being affected to accommodate the insulin molecules, thus losing crystallinity.

TABLE 1
Physicochemical characterizations of TTA-DFP-nCOF before and
after insulin loading.

| | TTA-DFP-nCOF | TTA-DFP-nCOF/Insulin |
|---|---|---|
| PXRD, 2θ (plane) | 4.9° (110), | 4.9° (110), |
| | 25.6° (003) | 25.6° (003) |
| AFM height, nm | 7 | 12 |
| TEM (width), nm | 124 | 147 |
| Hydrodynamic radius, nm (PD) | 68 (0.1) | 91 (0.2) |
| ζ-potential, mV | ~−16 | ~−17 |
| BET, m²g⁻¹ (TPV, m³g⁻¹) | 385 (0.50) | 12 (0.02) |

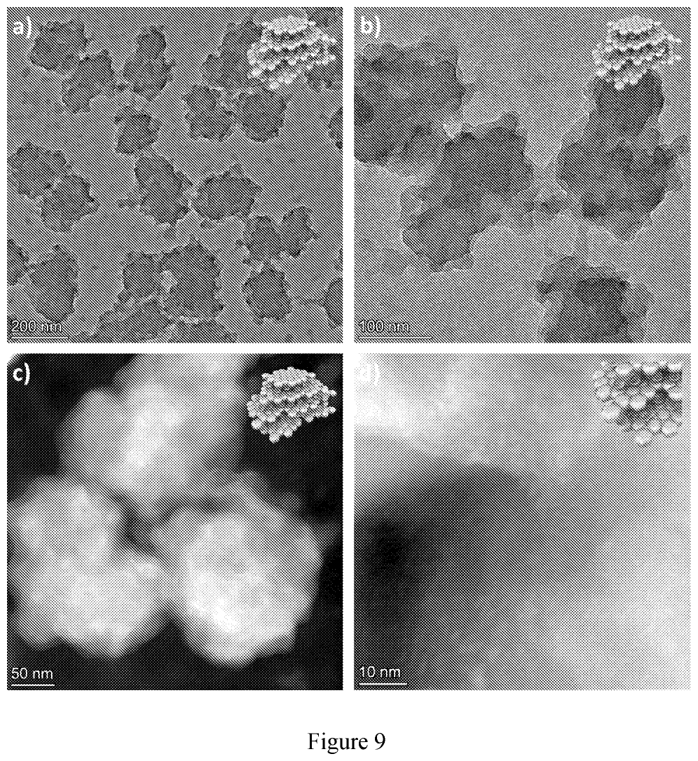

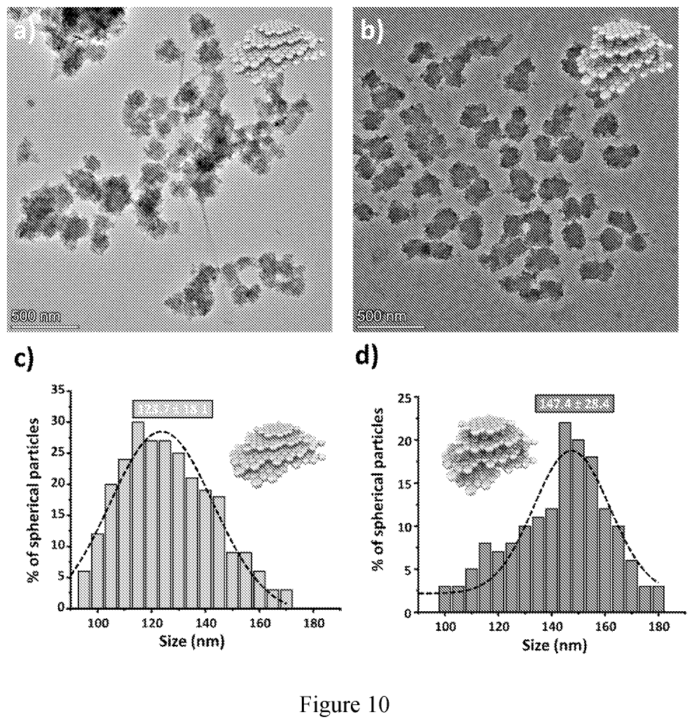

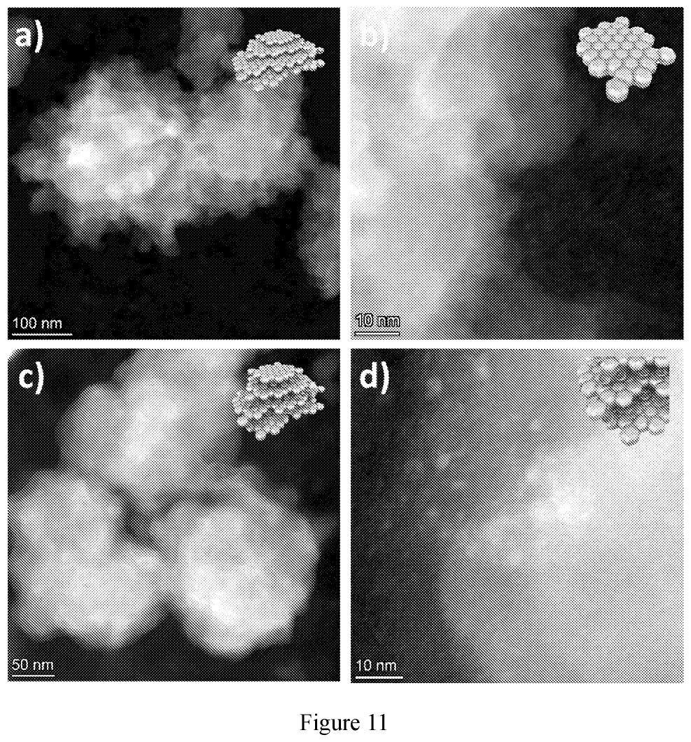

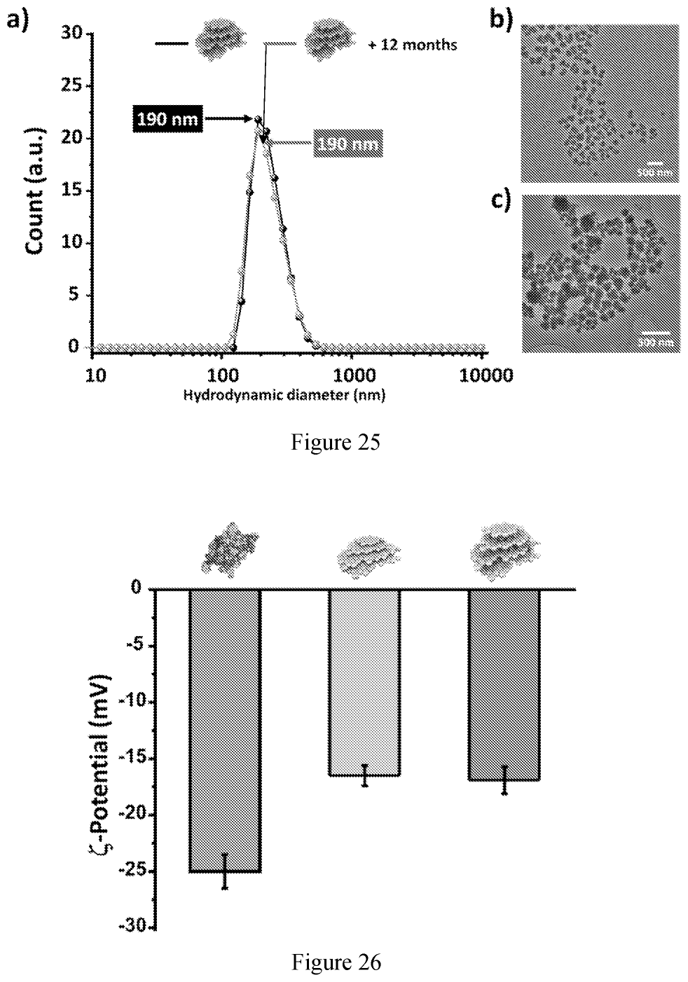

The uniformity of TTA-DFP-nCOF/Insulin is evident from AFM analysis which increased to 12 nm upon insulin encapsulation compared to pristine nCOF (FIG. 13, Table 1). AFM images also show an increase in nanoparticle width upon insulin loading, corroborated with TEM (FIGS. 1e and 9-11, Table 1). This suggested a slipping of nanosheets to accommodate the insulin molecules, a fact that is supported by the loss of crystallinity shown by PXRD. The hydrodynamic radii of nanoparticles without and with insulin loading shows an increase in solvated particle size as well as an increase in polydispersity from narrowly monodisperse to moderately polydisperse, showing a slight distribution in insulin encapsulation (Table 1, FIG. 25). ζ-potential measurement shows no statistically significant difference pre- and post-insulin loaded nCOF yet they are both markedly different from insulin on its own, strongly supporting insulin encapsulation within the nanoparticles, rather than on its surface (Table 1, FIG. 26). Following insulin loading, N₂ sorption (FIG. 16) displays a Type IV isotherm, typical of mesoporous materials, with a low-pressure H4-type hysteresis which either indicates swelling of a non-rigid porous structure or that insufficient equilibration is achieved during measurements because of slow N₂ diffusion in ultramicropores or that significant micropores exist but whose access is blocked.

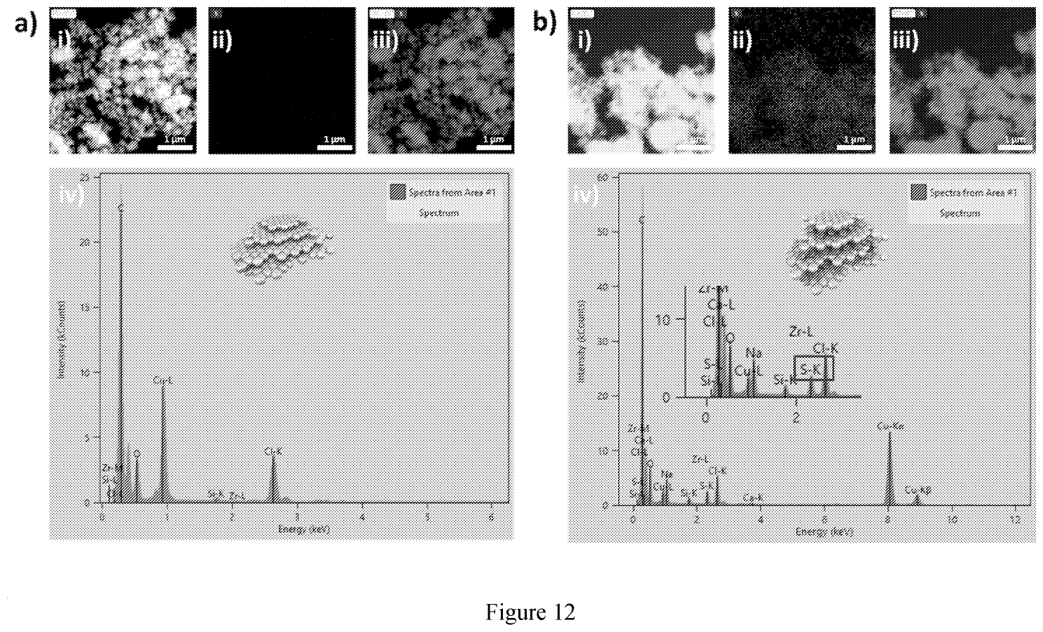

Further evidence for insulin-loading between the nanosheets versus inclusion or diffusion through the micropore channels formed by stacking of nCOF layers derives from the size of the protein molecules (2.5-3 nm) as compared to the diameter of the micropore channels (1.7 nm, FIG. 16). Insulin molecules are likely intercalated between several layers, favored by the small size of the TTA-DFP-nCOF particles. Simulation of the adsorption of insulin molecules between TTA-DFP-nCOF layers was performed following a simulated annealing process, where one insulin monomer was included between two sets of three ABC-stacked layers (FIG. 1g). A loading amount corresponding to ˜ 70 wt % was calculated which is comparable to the maximum loading achieved experimentally (Calculation details below). To accommodate the insulin molecules, each set of layers were separated by 2.5 nm along the stacking direction during the simulation process. The resulting minimum energy conformation displayed in FIG. 1g shows how the insulin molecules are accommodated and interact with the nCOF layer atoms, which is also favored by the presence of pores, where some of the insulin terminal peptides pointed. TEM dispersive X-ray spectroscopy (TEM-EDX) indicates that insulin is uniformly distributed throughout TTA-DFP-nCOF (FIG. 12).

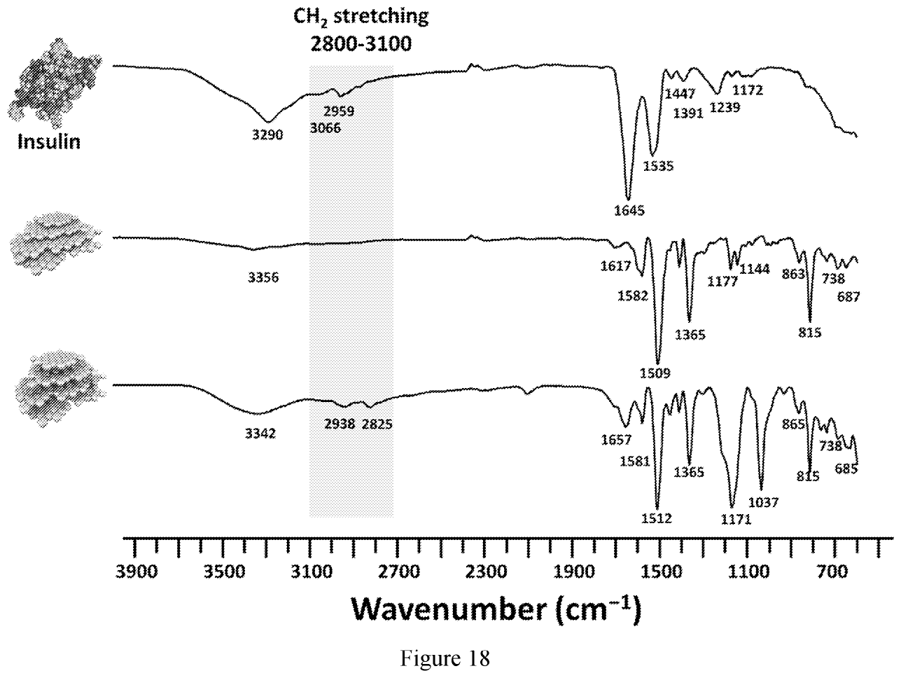

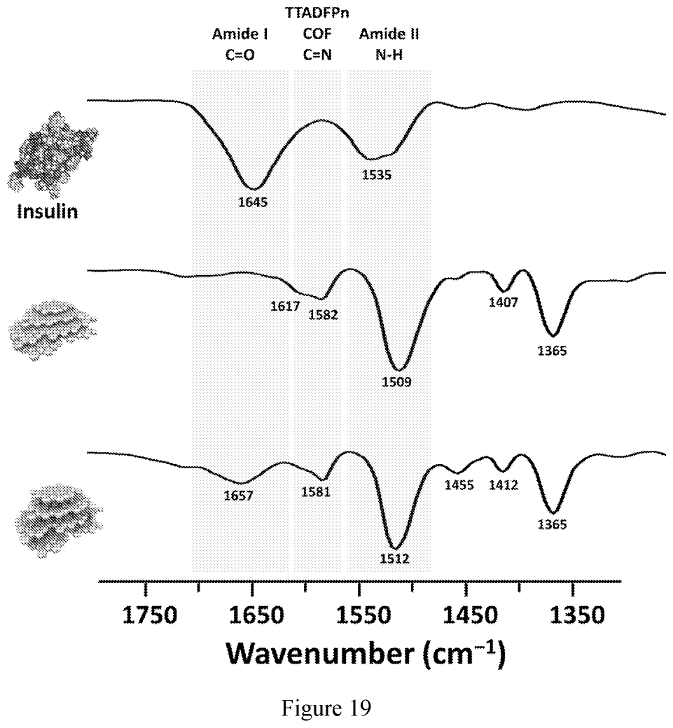

To examine the TTA-DFP-nCOF and insulin interactions, Fourier-transform infrared spectroscopy (FTIR), and X-ray photoelectron spectroscopy (XPS) spectroscopy analysis were employed. The FT-IR spectrum of insulin displayed characteristic protein peaks: i) at 2800-3000 cm⁻¹ corresponding to CH₂ stretching bond, ii) at 1645 cm⁻¹ for amide I and iii) another at 1535 cm⁻¹ corresponding to amide II mainly due to C═O stretching vibration (FIGS. 18-19). After insulin entrapment in TTA-DFP-nCOF, a new set of peaks were observed in the spectrum of TTA-DFP-nCOF/Insulin at 2800-3000 cm⁻¹ and at 1657 cm 1 corresponding to CH₂ stretching bond and C═O Amide I of the insulin, respectively, with significant shifts (FIGS. 18-19). The peak at 1535 cm⁻¹ is overlapping with the TTA-DFP-nCOF C═C aromatic bonds. In addition, the disappearance of the imine bond at 1617 cm⁻¹ of the COF was also observed. These observations could to be associated with weak insulin-TTA-DFP-nCOF interactions between amide I C═O group of the insulin and the COF imine bond.

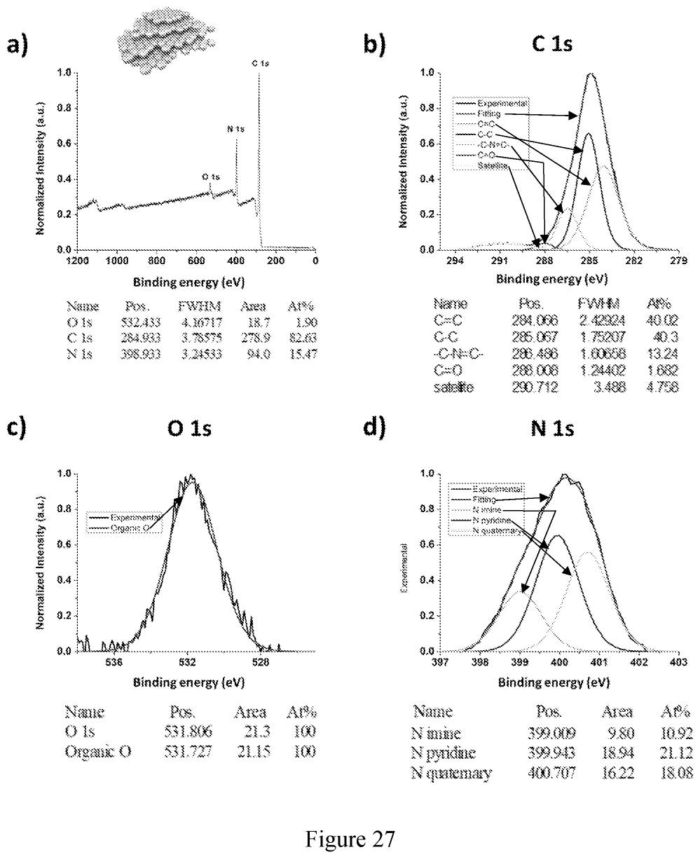

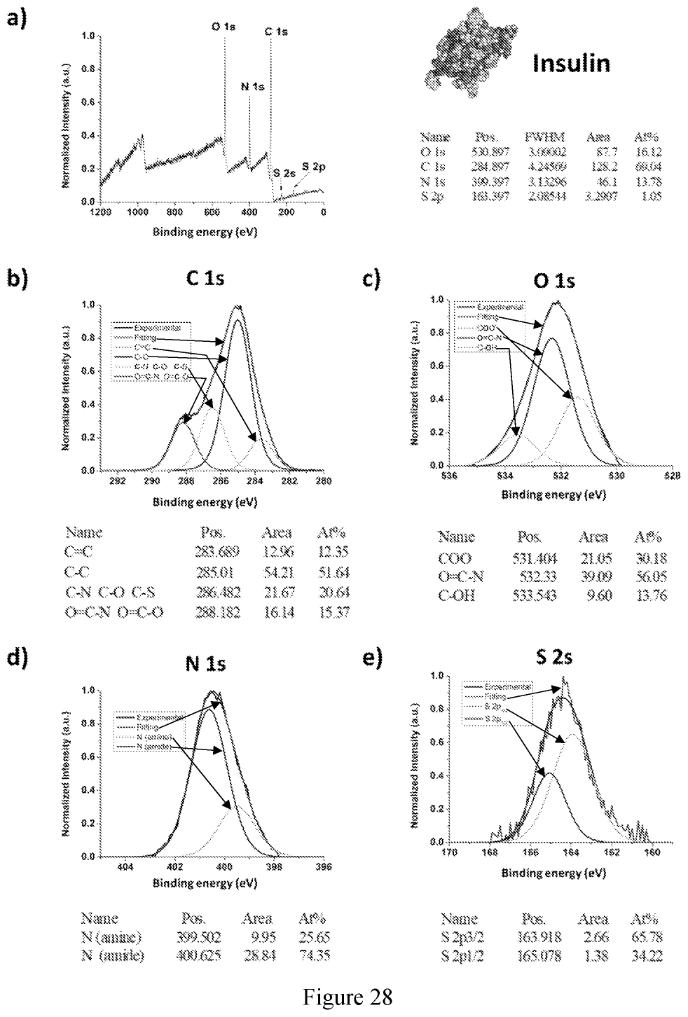

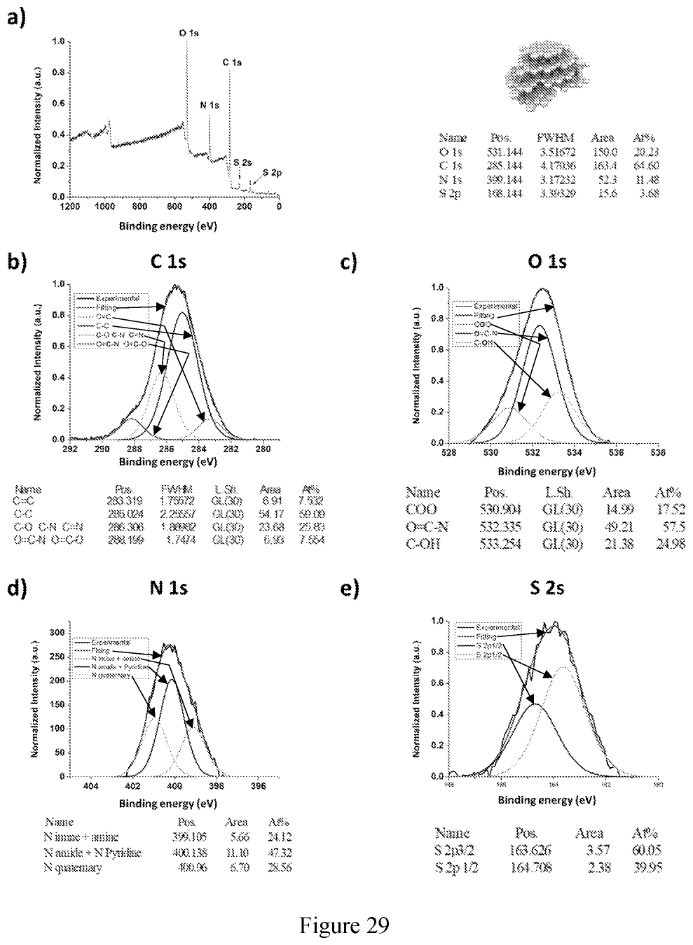

Moreover, XPS analysis was performed in order to understand the interactions occurring between the nCOF and the protein. The presence of sulfur beside the net increase of oxygen (around 10-fold) in the XPS survey spectrum of the TTA-DFP-nCOF/Insulin compared with the survey spectrum of TTA-DFP-nCOF represent a clear proof of insulin presence in the nanoparticles (FIGS. 27-29). Indeed, sulfur is present in insulin due to cysteine amino acids and is absent in nCOF (FIG. 28). The C1s high resolution XPS spectrum in TTA-DFP-nCOF/Insulin (FIG. 29) comprises four peaks at 283.4, 285.1, 286.3 and 288.2 eV attributed to the C═C sp², C—C sp³, C—O/C—N bonds and carbon in carboxylic or amide groups, respectively. A significant increase in C sp³ percentage could be observed in the deconvoluted peak C Is of TTA-DFP-nCOF/Insulin compared with C Is of the nCOF (FIG. 27-30). This increase could be attributed to the contribution of C sp³ present in the side chains of amino acid that exist in the structure of insulin. FIG. 29 displays the O 1s XPS survey spectrum in TTA-DFP-nCOF, where the peak is composed of three components centered at 531.4, 532.3 and 533.5 eV attributed to oxygen in carboxylate, amide and alcohol groups respectively. After loading TTA-DFP-nCOF with insulin, an important decrease (around 12%) in the percentage of oxygen assigned to carboxylate was observed (FIG. 28c). Deconvoluted N Is spectrum of TTA-DFP-nCOF (FIG. 27d), indicates the presence of three distinct peaks at (i) 399.0 eV corresponding to the imine nitrogen, (ii) 399.9 eV associated with pyridinic nitrogen, and (iii) 400.7 eV attributed to quaternary nitrogen. On the other hand, the N Is spectrum for the mixture TTA-DFP-nCOF/Insulin exhibits the contribution of nitrogen from amino and amide groups present in insulin (FIG. 27d). Additionally, a decrease in the percentage of the quaternary amine (around 8%) in the N Is peak of the mixture compared with TTA-DFP-nCOF was observed. Both together, the decrease in the percentage of oxygen assigned to carboxylate existing in insulin and the percentage of quaternary amine existing in TTA-DFP-nCOF strongly indicate that the interactions occur between the carboxylate of insulin and the quaternary amino groups of TTA-DFP-nCOF.

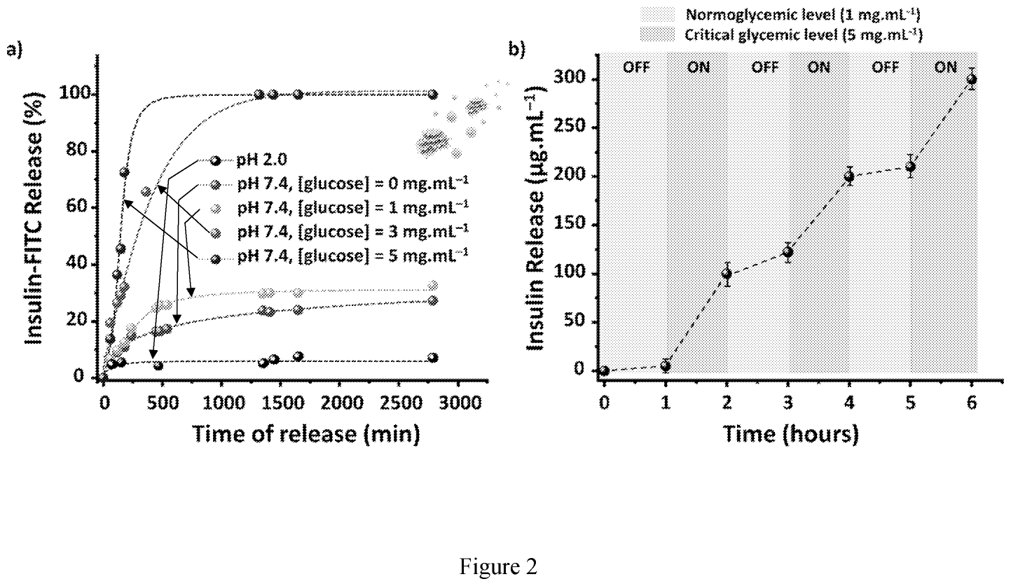

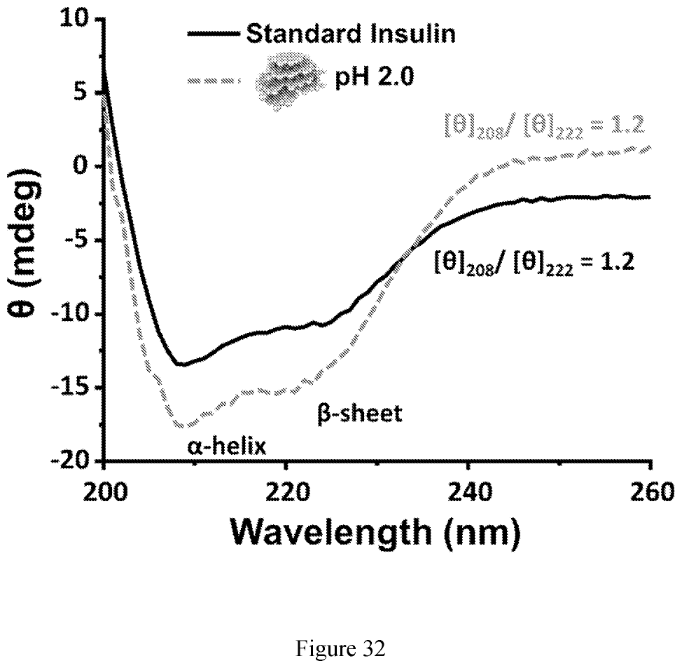

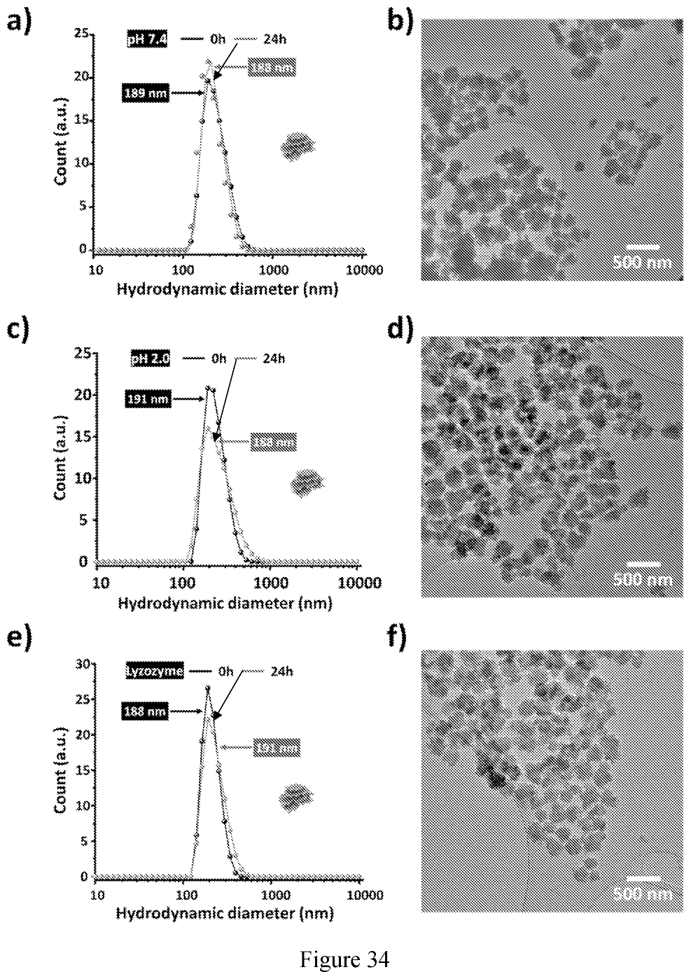

The efficacy of TTA-DFP-nCOF/Insulin-FITC in vitro was tested against both simulated gastric and intestinal fluids (SGF, pH 2.0; SIF, pH 7.4). Quantification of insulin-FITC release was carried out using fluorescence spectroscopy and experiments showed insignificant insulin release in both SGF (<5%) and SIF (<15%) following 24 h incubation (FIG. 2a). The circular dichroism (CD) structure of the insulin after being emerged in acidic conditions inside the nanoparticle was not affected, displaying the same pattern as the native insulin (FIG. 32). Moreover, in order to confirm the stability of the TTA-DFP-nCOF/Insulin, a DLS study at pH 7.4, 2.0 and in presence of lysozyme (enzyme abundant in secretions including tears, saliva, and mucus) over a period of 24 hours was performed, followed by TEM images (FIG. 34). The size and morphology of the TTA-DFP-nCOF/insulin does not vary in the various condition mimicking the stomach. Confinement of insulin between the nanosheets of the nCOF nanoparticle protects insulin from unfolding and degradation thus offering protection necessary for oral delivery.

Evaluation of the in vitro capacity for TTA-DFP-nCOF/Insulin-FITC to respond to a hyperglycemic-triggered drug release was investigated against various glucose concentrations; control, normal and diabetic, with [glucose]=0, 1, 3 and 5 mg mL⁻¹, respectively, in PBS (10 mM, pH=7.4). Under control conditions, only 12% of insulin was released over a 24 h incubation period, rising to 28% for normoglycemia, showing a slow, natural release of insulin. Importantly, however, under hyperglycemic conditions exhibited by diabetic patients (3 mg mL⁻¹) there was almost 100% insulin release after 7.5 h incubation (FIG. 2a).

With glucose levels naturally fluctuating between hunger and satiation, the on-off regulation of insulin release was monitored between a normal and hyperglycemic state every 1 h, with the insulin release profile of TTA-DFP-nCOF/Insulin-FITC displaying a pulsatile pattern (FIG. 2b).

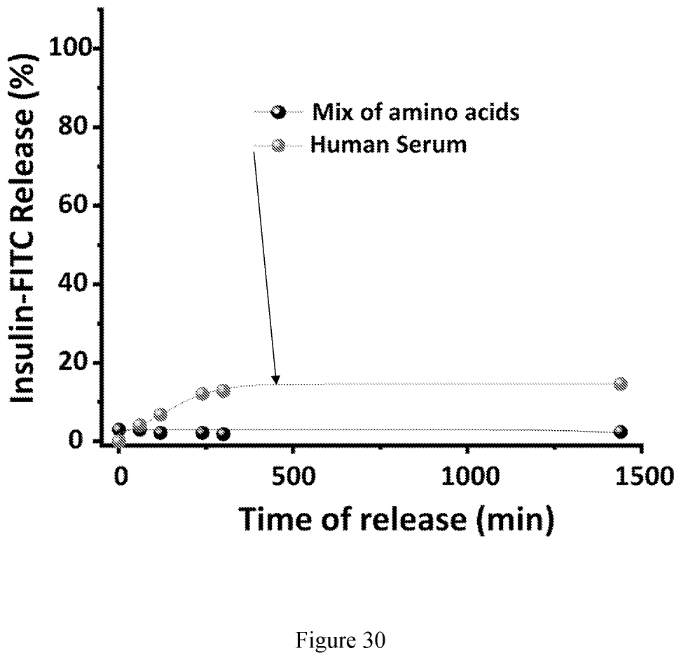

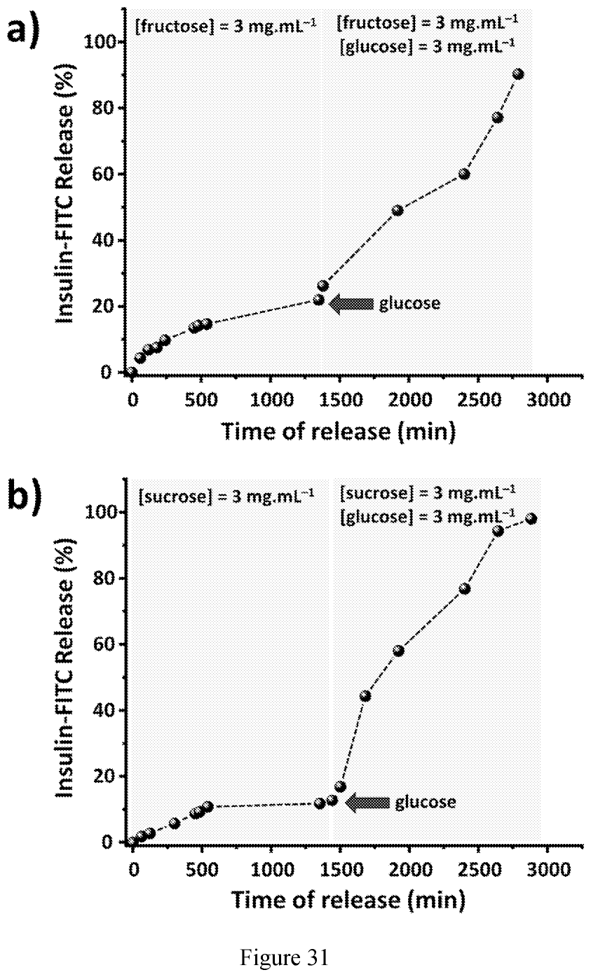

To confirm insulin release is specifically triggered by glucose, TTA-DFP-nCOF/Insulin-FITC was incubated at 37° C. for 24 hours in either human serum, a mix of 11 amino acids, a saline solution of fructose (3 mg mL⁻¹) or sucrose (3 mg mL⁻¹; FIGS. 30-31). There was negligible release of insulin observed up to 24 h in all the conditions tested with maximum insulin-FITC released of 15% in human serum, 3% in amino acids, 22% in fructose, and 13% in sucrose. However, after incubation with fructose or sucrose, the glucose concentration was adjusted to mimic hyperglycemia (3 mg mL⁻¹), a burst release of insulin was observed, confirming the specific glucose-responsiveness of this nCOF system.

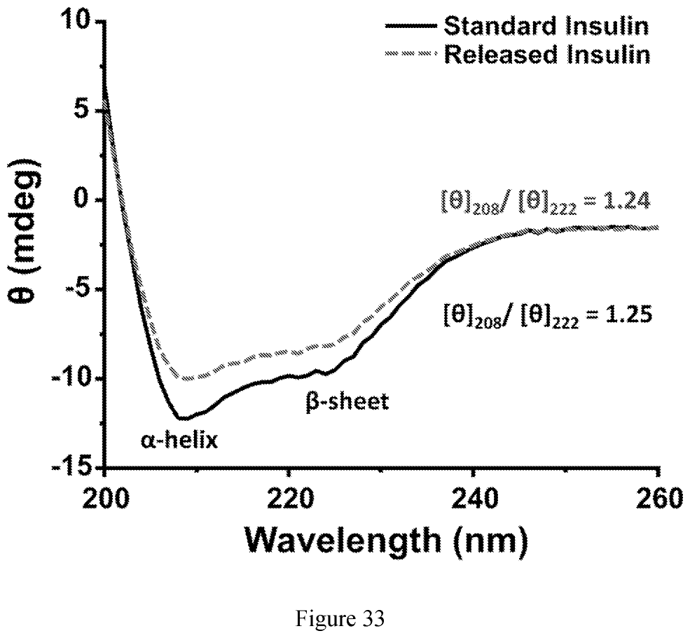

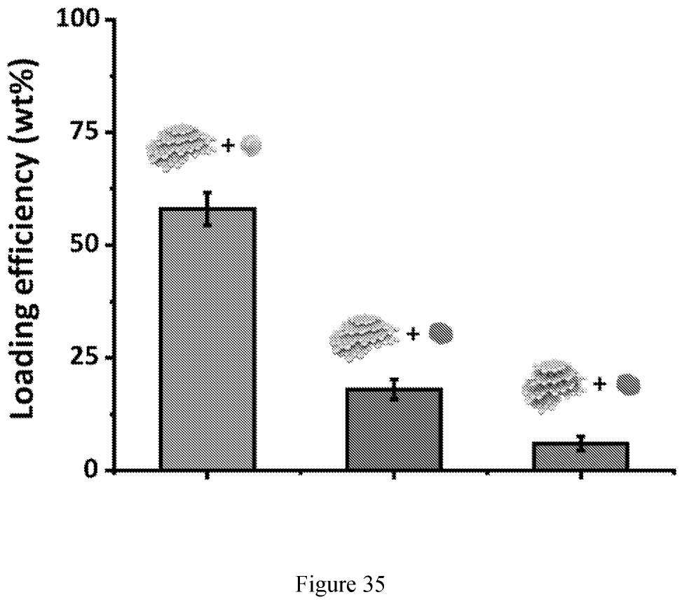

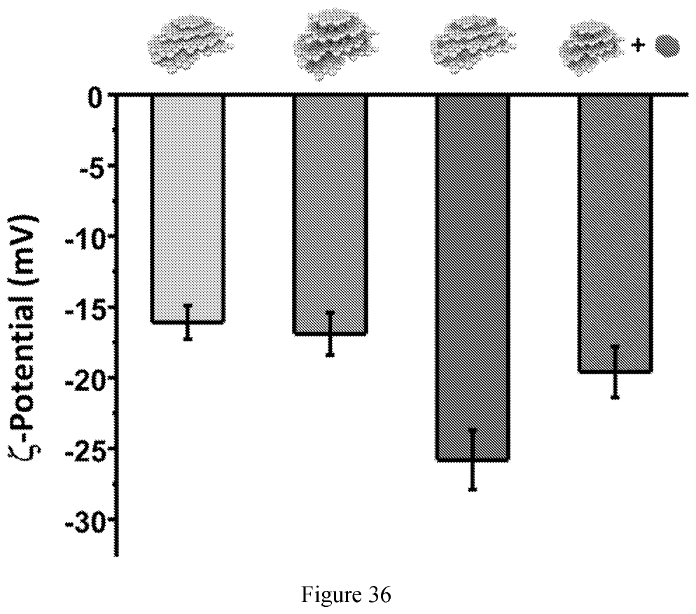

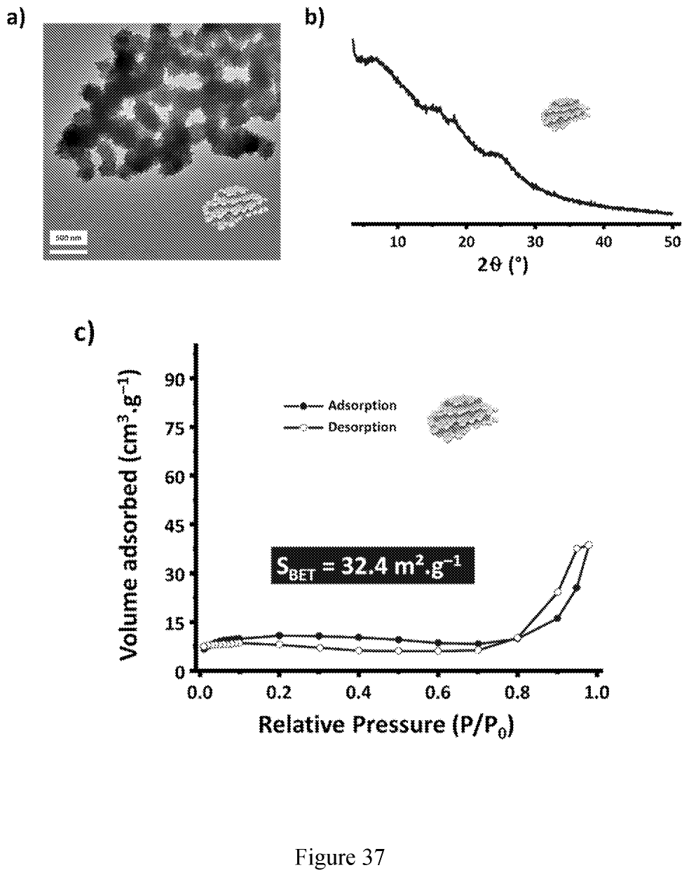

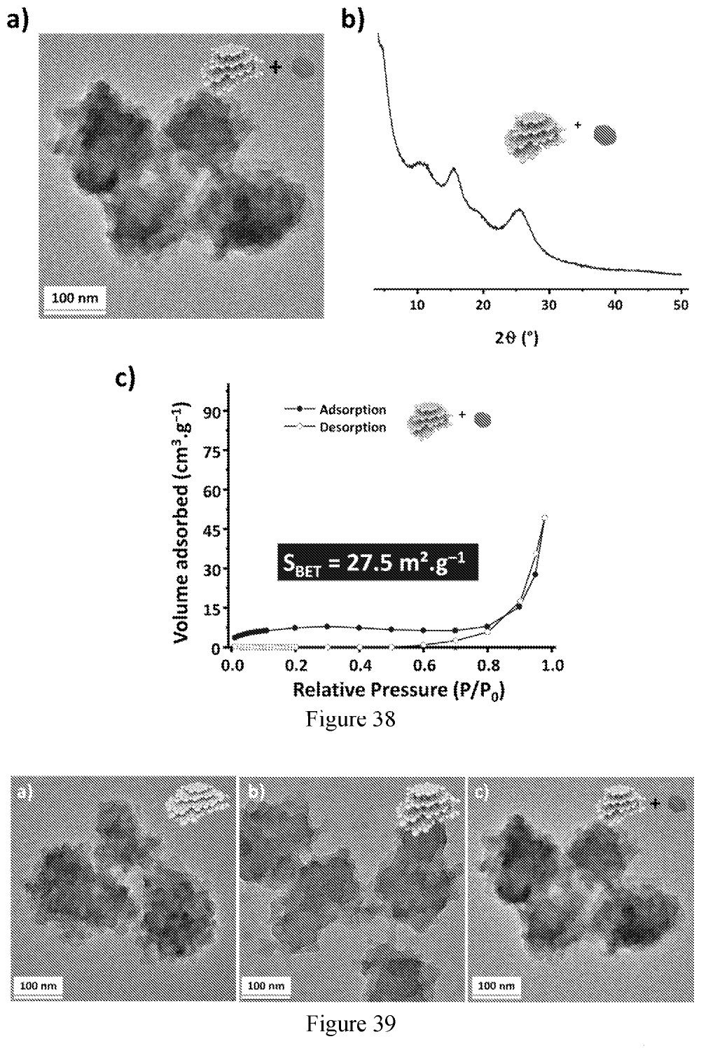

Compared to the size of insulin (d=2.5-3.0 nm), glucose is a small molecule (d=0.8 nm) that can fit inside the nCOF pores (1.7 nm). The TTA-DFP-nCOF can take up to 18 wt % of glucose (24 h, 5 mg·mL⁻¹) due to hydrogen bonding between the numerous hydroxyl groups and the nitrogen atoms of the framework (FIGS. 35-38). Under hyperglycemic conditions, glucose is forcefully diffused through the micropores of nCOF displacing insulin from between the nanosheets. After 24 hour hyperglycemic interactions, TTA-DFP-nCOF were found almost empty (loading efficiency 6 wt %). TTA-DFP-nCOF/glucose displayed a charge of −25.8 mV due to the large presence of hydroxyl functions of glucose on the surface and within the framework (FIG. 36). TTA-DFP-nCOF/Insulin followed with glucose incubation displayed a charge of −19.6 mV. This strongly indicates a glucose concentration dependence of insulin displacement. The size of glucose means preferential filling of nCOF pores, too small for insulin. However, once these pores are filled at normoglycemic concentrations, increasing glucose concentrations means glucose molecules start to penetrate between the nanosheets, thereby displacing the insulin, forcing it out of the nanoparticle. However, the extent of glucose absorption into the micropores of the nCOF do not constitute an obstacle to the insulin oral delivery. The TTA-DFP-nCOF can take up a maximum of 18 wt % of glucose in unrealistic hyperglycemic conditions which most likely will not happened in a patient. Therefore, it is assumed that the TTA-DFP-nCOF/Insulin will not lead to a hypoglycemic state by dropping the glucose levels too quickly in vivo or in a patient. TEM, PXRD and BET after Insulin release in hyperglycemic conditions showed a decrease in size as well better crystallinity and increased BET surface compared to TTA-DFP-nCOF/Insulin (FIGS. 38-39). The preservation of insulin's secondary structure following release from the nCOF nanoparticle was assessed using circular dichroism and was found similar to that of the original insulin (FIG. 33); thus, nCOF-encapsulated insulin maintains both its structure and properties during transport and release, and, with this system exhibiting a glucose-triggered release mechanism, is an ideal candidate for the treatment of diabetes.

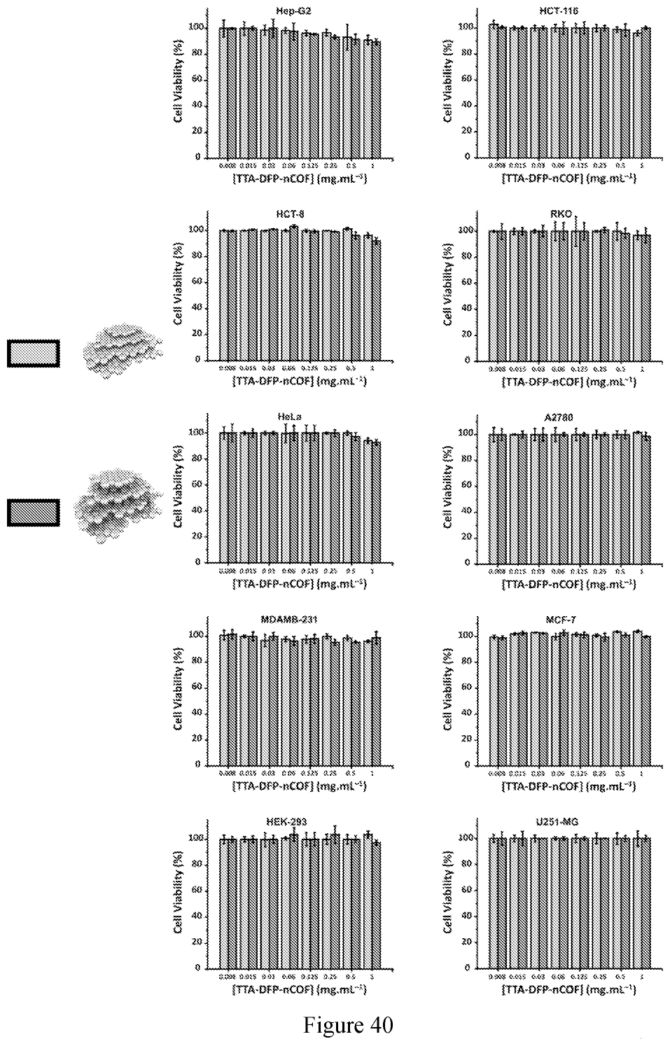

In vitro viability studies were carried out on 10 different cell lines (liver, colon, cervix, ovary, breast, kidney and brain, FIG. 40) to demonstrate the potential of TTA-DFP-nCOF as a biocompatible delivery vehicle. Both TTA-DFP-nCOF and TTA-DFP-nCOF/Insulin elicited no cytotoxic effects at TTA-DFP-nCOF concentrations up to 1 mg mL⁻¹ following 48 h of incubation indicating excellent biocompatibility and, therefore, had great potential for oral application.

TEM was used to investigate the effects of TTA-DFP-nCOF/Insulin on cellular structures and their interactions with organelles on 2 colon cell lines (RKO and HCT-116, 4-hour incubation times) and analysed 4 h, 24 h and 48 h post-treatment (50 μg·mL⁻¹; FIG. 41-43) since the material is aimed to cross the intestinal barrier. All the TTA-DFP-nCOF/Insulin treated samples showed the regular ultrastructure of the RKO and HCT-116 cells, with a roundish cellular shape and a plasma membrane rich in protrusions (such as microvilli), a well-developed rough endoplasmic reticulum, Golgi apparatus, and mitochondria, which indicate the maintenance of metabolic active cells. Significant amounts of TTA-DFP-nCOF/Insulin can be visualized within some of the treated cells and at their surface. Membrane deformation was also observed, confirming the internalization of TTA-DFP-nCOF/Insulin by endocytosis. Overtime, in both cell lines the TTA-DFP-nCOF/Insulin could be found inside cell vacuoles in the perinuclear region but no more on the membrane; cells continue growing and dividing, confirming that TTA-DFP-nCOF/Insulin is deemed non-toxic and safe with no deleterious effect on cell morphology, viability, mitochondrial health and did not lead to the production of any reactive oxygen species.

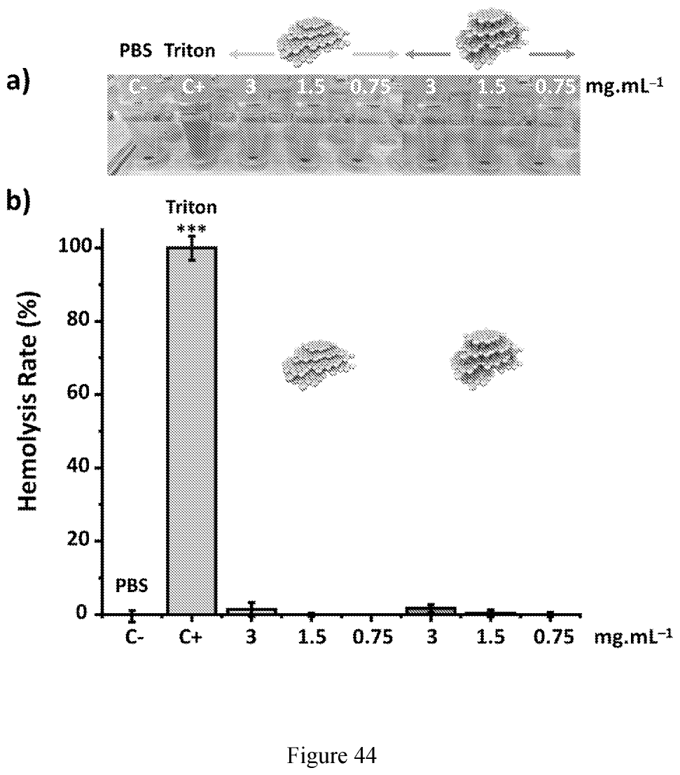

Hemolysis assay were carried out to determine biocompatibility and non-immunotoxicity of the material to human erythrocytes since the material is aim to penetrate the bloodstream. The results of hemolytic test (FIG. 44) demonstrated that the hemolytic rate (HR) of the samples were lower than 2%. According to ASTM F 756-08 (Standard Practice for Assessment of Hemolytic Properties of Materials), HR<5% produced by any material could be considered as not hemolytic, therefore TTA-DFP-nCOF and TTA-DFP-nCOF/insulin are biocompatible with human blood erythrocytes giving preliminary results on their lack of immunotoxicity. The charge of the nanoparticle is a critical parameter in the hemolytic response, nanoparticle with negative surface charge not being hemolytic explaining these results. It was suggested that positively charged particles caused them to act as surfactants and resulted in erythrocyte membrane disruption.

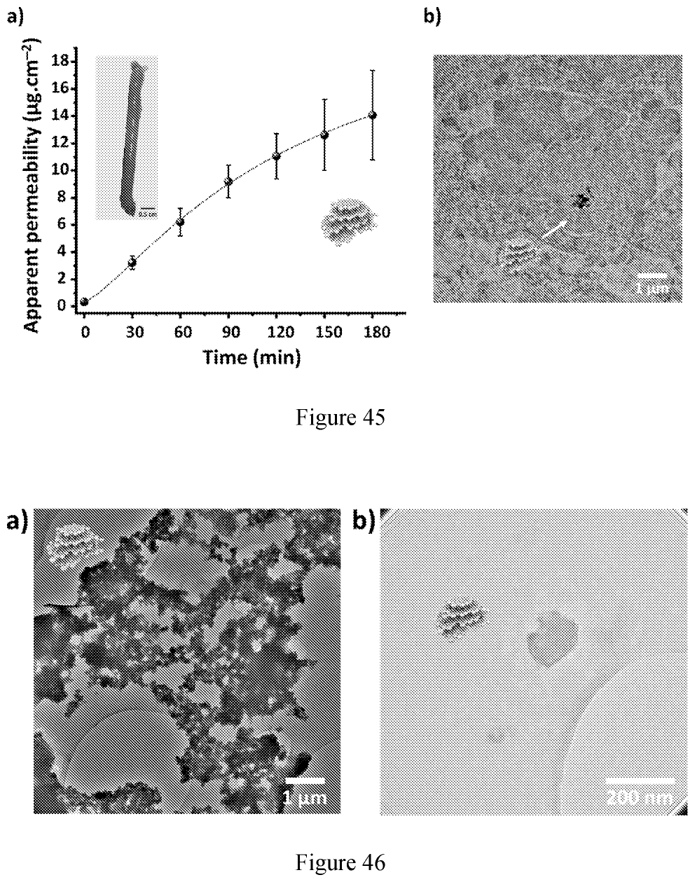

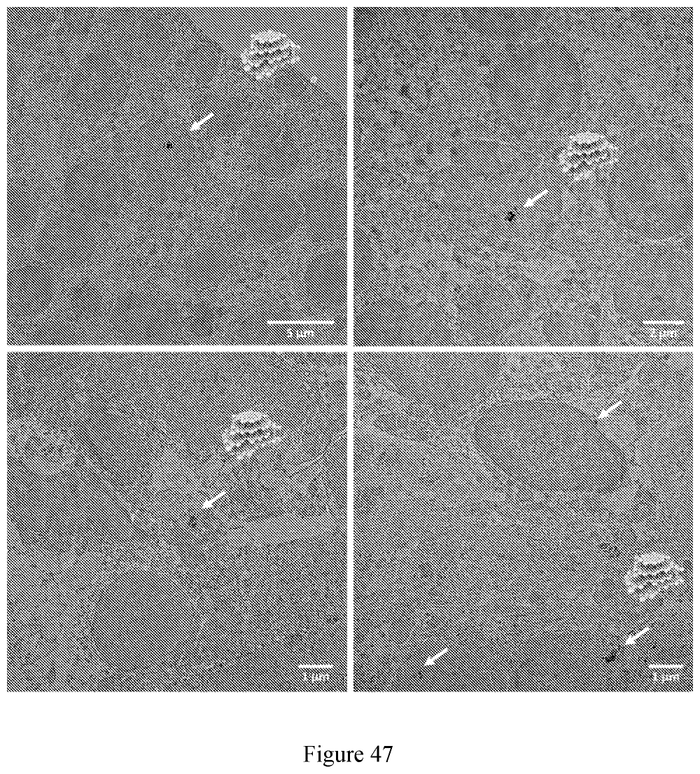

The ability of the TTA-DFP-nCOF/Insulin to cross the intestinal barrier was assessed during ex vivo experiments (FIG. 45). It was reported that the intestinal uptake of therapeutic proteins through nanoparticles regulated by particle size can improve the transporting ability of proteins through the epithelial lining of the small intestine while protecting the proteins against degradation in gastric fluid. TTA-DFP-nCOF/Insulin-FITC transportation across the intestine was assessed by measuring the apparent permeability using an ex vivo technique in excised rat small intestine using non-everted mouse small intestine sac model. The transportation of TTA-DFP-nCOF/Insulin was measured from the mucosal side to the serosa side of the non-everted mouse small intestine sacs and quantified by fluorescence measurements. Permeability of TTA-DFP-nCOF/Insulin after 3 h was calculated to be 14.76 μg·cm⁻² (corresponding to 60.8%±14.2 of the initial dose), while pure insulin was reported to be 8.02 μg·cm⁻². This indicates that incorporation of insulin into TTA-DFP-nCOF resulted in approximately two fold increase in permeability of insulin. Permeation data correlate with accumulation in the gut wall. This can be possibly attributed to enhancement of surface area leading to a higher rate of insulin-FITC diffusion. The serosa side was collected and TEM images was performed confirming that the TTA-DFP-nCOF/Insulin-FITC crossed the intestine and not free insulin-FITC. As shown in FIG. 46, TTA-DFP-nCOF/Insulin-FITC were present without modification of their morphologies and sizes in the serosa side. This accumulation may result from nanoparticles potential cellular internalization. Therefore, at the end of the experiments, tissues were washed with normal saline, and NP accumulation in the gut wall was investigated by TEM of the intestinal tissue (FIG. 47). TEM images of the intestine sections show their morphology with intact microvilli and underlying architecture of the ileal mucosa. TTA-DFP-nCOF/Insulin were located inside the goblet cells (GCs) of the whole intestinal tissue and were excreted into the gut lumen through the secretion of intestinal GC. Altogether it confirmed that TTA-DFP-nCOF/Insulin can cross the intestinal barrier carrying their insulin cargo and did not cause obvious pathological changes in intestinal tissues.

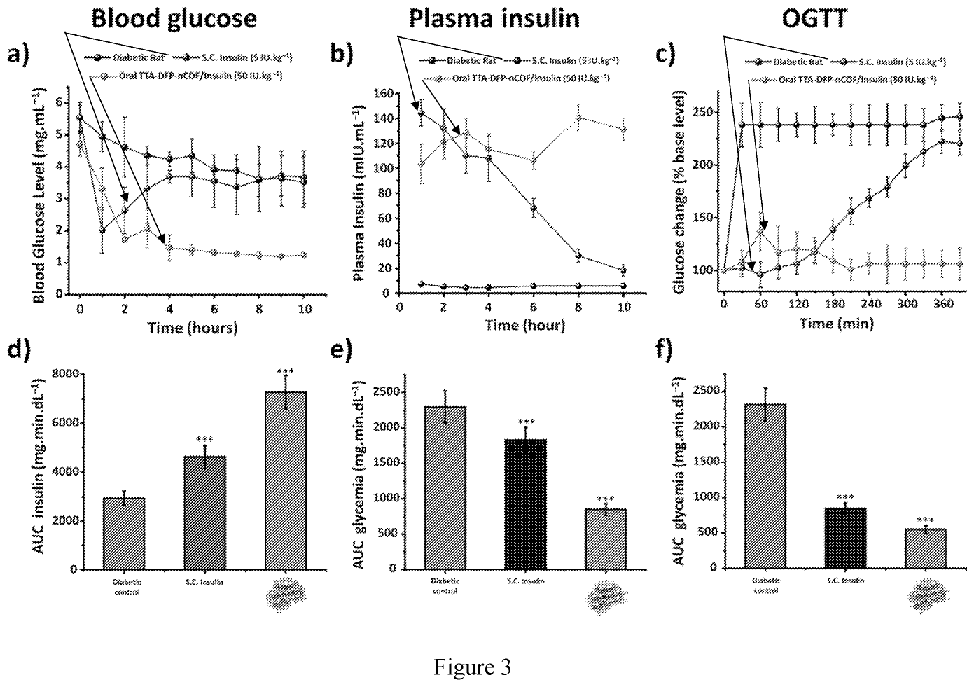

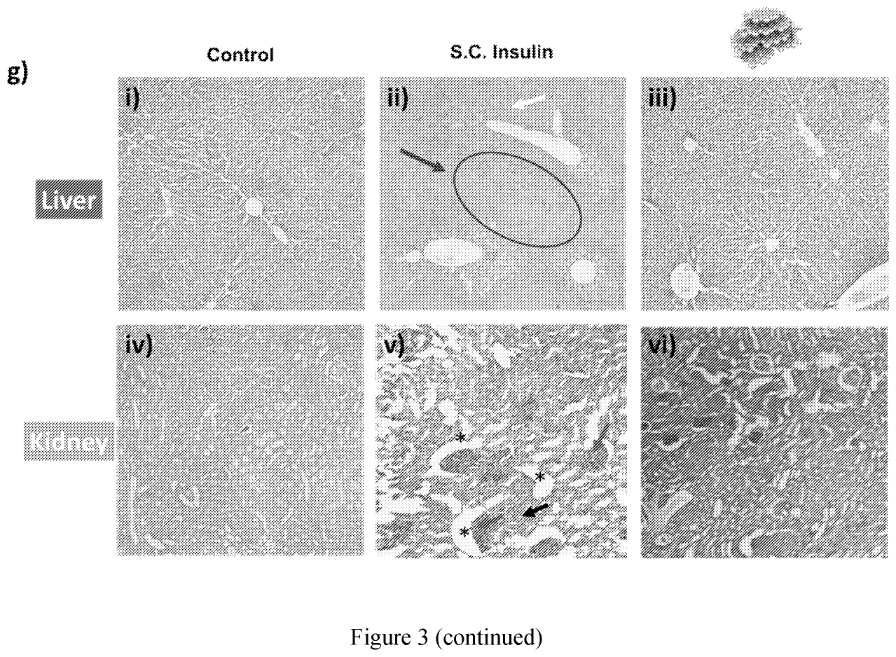

The in vivo pharmacological effect of the glucose-responsive TTA-DFP-nCOF/Insulin was evaluated by oral administration to a streptozotocin(STZ)-induced Type 1 diabetic (TID) rat model (FIGS. 3a-d and 48). The changes in blood glucose levels of the diabetic rats as a function of time following oral administration of various formulations or subcutaneous injections of free-form insulin solution were determined (FIGS. 3a-d and 48). Oral delivery of insulin-free TTA-DFP-nCOF (2 mg kg⁻¹) or free-form insulin solution (50 IU kg⁻¹) exhibited nearly no oral pharmacological availability as demonstrated by the minimal hypoglycemic effect observed. By contrast, subcutaneous injection of free-form insulin solution (5 IU kg⁻¹) resulted in a marked reduction of the blood glucose level within 1 h, though the effect was not sustained, with blood glucose levels rapidly returning to those similar to insulin-free nCOF. However, following oral administration of TTA-DFP-nCOF/Insulin (50 IU kg⁻¹), a significant reduction in blood glucose levels to those of non-diabetic rats was observed within 2 h coupled with a sustained hypoglycemic effect for 10 h, replicating the normal glucose level of the non-diabetic rat control group (FIG. 3a). These results are in concordance with the insulin plasma levels which showed that TTA-DFP-nCOF/Insulin-treated rats presented the highest levels of plasmatic insulin. Subcutaneous insulin rats showed a peak of insulin plasma level in the first hour after insulin injection, but insulin plasma levels rapidly decreased, as described in literature. Homeostatic model assessment (HOMA) is a method used to yield an estimate of insulin sensitivity, insulin resistance and β-cell function from fasting plasma insulin and glucose concentration. TTA-DFP-nCOF/Insulin-treated rats presented a lower HOMA-IR (insulin-resistance index) and a higher HOMA-IS (insulin-sensibility index) than the subcutaneous insulin rats, suggesting that the TTA-DFP-nCOF/Insulin particles are better assimilated by the body than the subcutaneous insulin (Table 2). Oral insulin is directly absorbed by the intestinal epithelium and reaches the liver through the portal vein, allowing maintenance of glucose homeostasis. Whereas parenteral administration of insulin never mimics the natural secretion of insulin as it is first delivered to peripheral tissues. Calculation of the bioavailability of TTA-DFP-nCOF/Insulin shows that TTA-DFP-nCOF/Insulin particles presents a high bioavailability (24.1%) comparing to insulin-loaded particles described in literature. This is in concordance with the insulin and glucose plasmatic levels observed above.

TABLE 2
Measurement by homeostatic model assessment (HOMA) of insulin
resistance (HOMA-IR) and insulin-sensibility (HOMA-IS) of β-cell
function using the changes in insulin and glucose concentrations after
subcutaneous insulin and TTA-DFP-nCOF/Insulin treatment of diabetic rats.

| | SC insulin | TTA-DFP-nCOF/Insulin |
|---|---|---|
| HOMA-IR | 0.6 (0.3) | 0.5 (0.1) |
| HOMA-IS | 50.4 (11.0) | 54.4 (13.2) |

Oral glucose tolerance test (OGTT) was used to assess the ability of the body to up-take the glucose in post-prandial conditions and to evaluate the sensibility of the body to endogenous insulin. Animals have firstly received the TTA-DFP-nCOF/Insulin by gavage or the subcutaneous insulin injection. 3 hours after that which coincide with the serum insulin peak previously observed, the animals received 2.5 g·kg⁻¹ of glucose dissolved 1 ml in water, then glycaemia was evaluated over a period of 280 minutes (FIG. 3c-f). Glycaemia was compared to subcutaneous insulin and diabetic control. Subcutaneous insulin decreases severely the glucose plasma level the two first hours after the glucose gavage, as described in the literature. From 150 min, the glycaemia begins to increase until it almost reaches the control values. It is reported that recurrent hypoglycaemic episodes caused by the subcutaneous insulin therapy compromise the function and integrity of brain cells. However, the hypoglycaemic activity of the TTA-DFP-nCOF/Insulin was at first more moderated than the subcutaneous insulin due to the fact that the oral administration of insulin allows high concentrations of insulin in the portal vein, without sustained peripheral hyperinsulinemia, thereby preventing neuropathy and retinopathy. From 120 minutes, glycaemia was kept low and stabilizes.

Diabetes mellitus is often associated with alterations of kidney and renal functions in rats; therefore, the impact of TTA-DFP-nCOF/Insulin on these vital functions was studied. FIG. 3 displays the histopathological study of the liver and kidney to detect organ pathology in non-diabetic rats, SC insulin and the TTA-DFP-nCOF/Insulin treated rats. SC-insulin treated group display the commonly observed damages to the liver and kidney due to STZ administration to induce diabetes. Regarding the TTA-DFP-nCOF/Insulin treated rats, it can be seen that there is no damage caused to any of these organs, suggesting the TTA-DFP-nCOF/Insulin to be non-toxic but also proved that oral delivery of insulin can inhibit histopathological alterations induced by diabetes in rats.

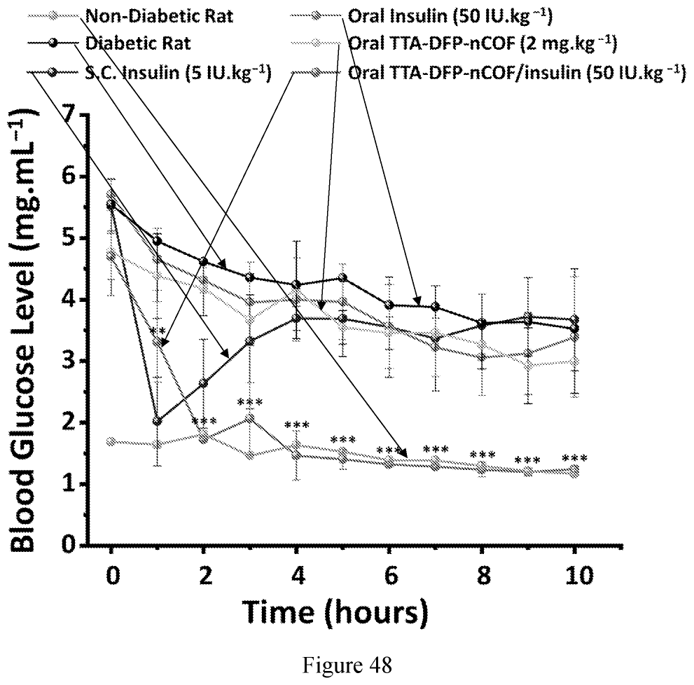

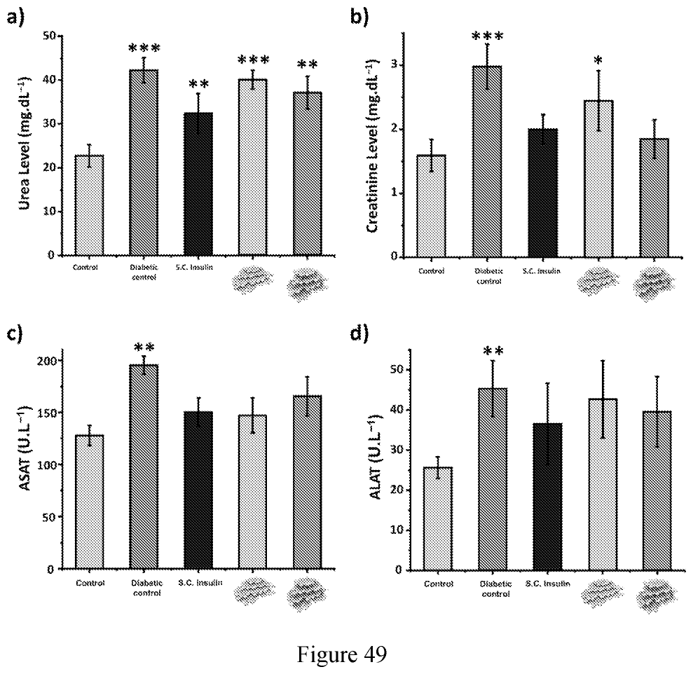

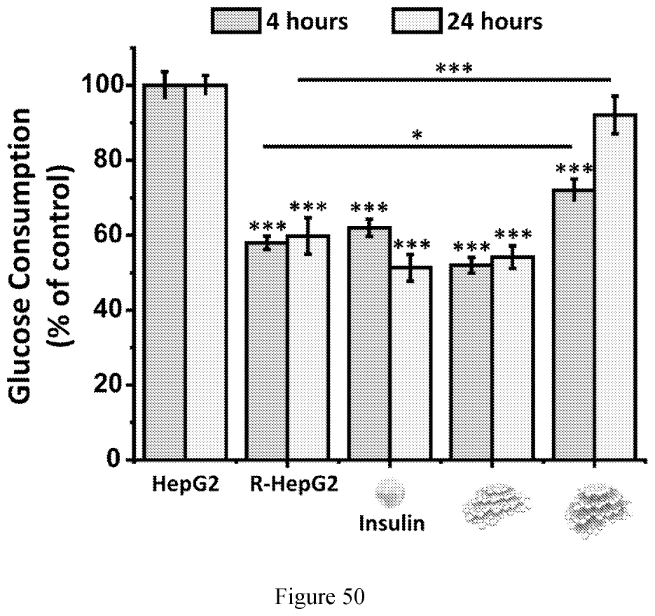

Furthermore, an exploration of biochemical markers for liver and kidney function in the blood can provide additional useful information to pick up the beginnings of toxic effects. Urea and creatinine are biomarkers commonly used to observe alterations in kidney function, while aspartate aminotransferase (ASAT) and alanine aminotransferase (ALAT) are biomarkers of liver damage, literature reporting elevations of these 4 parameters on diabetic rats (FIG. 48). Overall TTA-DFP-nCOF/Insulin-treated rats present similar levels of urea, creatinine, ASAT and ALAT to the non-diabetic group (FIG. 49), demonstrating that TTA-DFP-nCOF/Insulin particles are not only non-toxic, but can also enhance kidney and liver functions compared to the diabetic control. Insulin oral delivery is described for being beneficial to kidney and liver functions.

### Conclusions

In conclusion, a nanoscale imine-covalent organic framework (TTA-DFP-nCOF) as an oral insulin delivery system was prepared and tested, in vitro and in vivo. TTA-DFP-nCOF's crystalline and porous nature allows among the highest loading of insulin to be achieved, with evidence showing insulin is located between layers of the nCOF nanosheets, rather than in the porous channels. TTA-DFP-nCOF was proven to protect encapsulated insulin in vitro under harsh conditions mimicking that of the stomach environment, while the sustainable release of insulin was accomplished under hyperglycemic conditions; importantly, insulin maintained its activity upon release from TTA-DFP-nCOF/Insulin. The oral administration of TTA-DFP-nCOF/Insulin to STZ-induced diabetic rats led to a continuous decline in the fasting blood glucose level within 2 to 4 h, and the hypoglycemic effect maintained over 10 h in vivo showing high insulin bioavailibity without systemic toxicity. The potential for this TTA-DFP-nCOF based insulin oral delivery system to replace traditional subcutaneous injections and enhance the uptake of insulin therapy amongst those in need has been demonstrated.

### Reagents and Techniques

All reagents and starting materials were purchased from Sigma-Aldrich and used without further purification. The precursor, 2,6-diformylpyridine (DFP) was synthesized according to the published procedure with no modifications. Deionized water was used from Millipore Gradient Milli-Q water purification system. Thin-layer chromatography (TLC) was performed on silica gel 60 F254 (E. Merck). The plates were inspected under the UV light. Column chromatography was performed on silica gel 60F (Merck 9385, 0.040-0.063 mm). Infrared spectra were recorded on an Agilent Technologies Cary 600 Series FTIR Spectrometer using the ATR mode. PXRD patterns of the samples were recorded by using an X-ray Panalytical Empyrean diffractometer. High resolution transmission electron microscopy (HRTEM) images were obtained using a Talos F200X Scanning/Transmission Electron Microscope (STEM) with a lattice-fringe resolution of 0.14 nm at an accelerating voltage of 200 kV equipped with CETA 16M camera. The high resolution images of periodic structures were analyzed using TIA software. N₂ adsorption-desorption isotherms were obtained at 77 K using Micrometrics ASAP 2020 surface area analyzer. The topography of the self-templated samples was analyzed by dynamic atomic force microscopy (5500 Atomic Force Microscope; Keysight Technologies Inc., Santa Rosa, CA). We acquired topography, phase and amplitude scans simultaneously. Silicon cantilevers (Nanosensors™, Neuchatel, Switzerland) with resonant frequencies of 250-300 kHz and force constants of 100-130 Nm⁻¹ were used. The set point value was kept at 2.5V. AFM scans were collected at 1024 points/lines with scan speed of 0.20 at fixed scan angle of 0°. Scan artifacts were minimized by acquiring a typical scan at an angle of 90° under identical image acquisition parameters. We used Gwyddion™ free software (version 2.47), an SPM data visualization and analysis tool for post-processing the AFM scans. Emission spectra in water at room temperature were recorded on a Perkin Elmer LS55 Fluorescence Spectrometer. Dynamic light scattering (DLS) measurements were performed on a Malvern Zetasizer NanoSeries to obtain the size and C-potential of the nanoparticles. The XPS experiments were carried out on a Kratos Axis Ultra DLD spectrometer under a base pressure of ˜2×10⁻¹⁰ mbar. A monochromated Al Kα X-ray source (1486.69 eV) was used to irradiate samples at room temperature. Far-UV spectra were recorded between 200 and 280 nm on a Chirascan CD spectrometer (Applied Photophysics, UK) with the lamp supplied with a flow of nitrogen. Fifty microlitres of the solution were added to a 0.1 mm path-length quartz cuvette (Hellma, UK) and the measurements were carried out at 20° C. (1 nm bandwidth resolution and 1 s acquisition time). Typically, at least two scans were recorded, and baseline and HEPES spectra were subtracted from each spectrum. Data were processed using Applied Photophysics Chirascan Viewer and Microsoft Excel. Phase contrast and fluorescence images were observed on an Olympus FV1000MPE confocal scanning microscope. Flow cytometry analyses were performed on Accuri C6 Flow Cytometer. The most favorable location of insulin molecules between COF layers was calculated with a simulated annealing process, using the Adsorption Locator module of Biovia Materials Studio. For this, the TTA-DFP COF structure was first modified by separating sets of three layers at a 25 Å distance, to allow the incorporation of insulin molecules. The insulin monomer was obtained from the 1zni structure of the protein data bank. One insulin monomer was incorporated per nCOF unit cell, which corresponds to ˜ 70 wt %. The Monte Carlo simulation was then run with the use of a universal forcefield after charge assignment, and the conformation with lowest adsorption energy was selected.

TABLE 3
Examples of COF materials loaded with enzymes in the
literature and their applications.

| COF | Enzyme | Results |
|---|---|---|
| TPMM COFs | Amylase | Catalytic |
| | | application in starch |
| | | hydrolysis |
| COF-42-B | Catalase, glucose | Enzymatic |
| | oxidase (GOx) | immobilization in |
| | | porous |
| | | matrixes |
| COF-1 and COF-5 | Insulin conjugated to | Glucose and pH |
| | glucose oxidase | dual-responsive |
| | (GOx) | insulin delivery |
| | | carriers, in vitro and |
| | | in vivo on diabetic |
| | | mice |
| TPB-DMTP-COF | Lysozyme | Understanding the |
| | | enzyme behavior |
| | | upon association |
| | | within |
| | | a confined space |
| COF-ETTA-EDDA | Lipase | Enzyme |
| | | immobilization |
| PPF-2 | Lipase | Enzyme |
| | | immobilization |
| TPB-DMTP-COF | Lipase | Host material for |
| | | the immobilization |
| | | and stabilization of |
| | | enzymes |
| COF-DhaTab | Trypsin | Enzyme |
| | | immobilization |

TABLE 4
Examples of insulin delivery systems and their loading capacities.

| | Material | Loading capacity |
|---|---|---|
| Insulin-loaded | Chitosan/poly γ- | 71.8% |
| chitosan | glutamic acid | |
| nanoparticles | | |
| Insulin-loaded | Alginate-chitosan | 56.7% |
| alginate | microspheres | |
| nanoparticles | Alginate | 75% |
| | microspheres | |
| Insulin-loaded | Alginate/dextran | 72.4% |
| dextran | sulfate (ADS)-NPs | |
| nanoparticles | | |
| Insulin-loaded | N-trimethyl chitosan | 47.0% |
| PLGA | chloride-coated | |
| nanoparticles | PLGA nanoparticles | |
| Insulin-loaded PLA | PLA-PEG | 42.7% |
| nanoparticles | microspheres | |
| Insulin-loaded PAA | Polyallylamine | 86.5% |
| nanoparticles | (PAA) grafted with | |
| | cholesteryl groups | |
| Insulin-loaded | Eudragit S100 ® | 80% |
| nanoparticles | (ES)-coated chitosan | |
| containing CPP | nanoparticles + | |
| | trans-activating | |
| | transcriptional | |
| | peptide (Tat) | |
| Lipid-Based | Solid lipid | 43% |
| Nanocarriers | nanoparticles | |
| | Nanostructured Lipid | 55.3% |
| | Carriers (NLCs) | |
| | Nanoemulsions | 47.3% |
| | water in oil in water | |
| | (w/o/w) | |
| Liposomes | Biotin-modified | 45% |
| | liposomes (BLPs) | |
| Niosomes | Span 60 niosomes | 28.8% |
| Micelles | Zwitterionic betaine | 6.2% |
| | polymer micelle | |
| Microgel | Glucose-responsive | 44.6% |
| | microgel | |
| Inorganic | Chondroitin sulfate | 90.1% |
| nanoparticles | (CS)-capped gold | |
| | nanoparticles | |
| | (AuNPs) | |
| | Silica nanoparticles | 7.4% |
| | Zirconium Phosphate | 28% |
| Metal-organic | MIL-100 | 35% |
| framework | ZIF-8 | 80.6% |
| | NU-1000 | 40% |
| | PCN-222/MOF-545 | 63% |

### Synthesis

Synthesis of 2,6-diformylpyridine (DFP) and 4,4′,4″-(1,3,5-triazine-2,4,6-triyl)trianiline (TTA). 2,6-diformylpyridine (DFP) was synthesized according to the methods known in the art with no modifications. 4,4′,4″-(1,3,5-triazine-2,4,6-triyl)trianiline (TTA): The triamine (TTA) was synthesized according to the methods known in the art with no modifications.

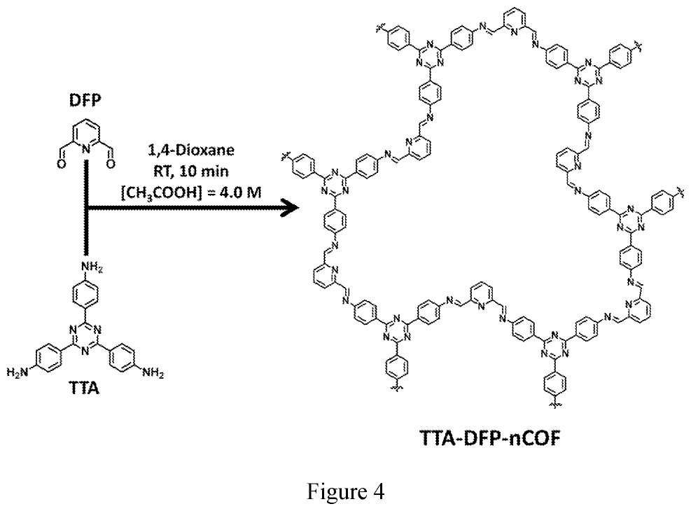

TTA-DFP-nCOF synthesis. TTA-DFP-nCOF was synthesized by co-condensation of 2,6-diformylpyridine (DFP, 21 mg, 0.15 mmol, 5 equivalents) and 4,4′,4″-(1,3,5-triazine-2,4,6-triyl)trianiline (TTA, 12 mg, 0.03 mmol, 1 equivalent), in 3 mL of anhydrous 1,4-dioxane in presence of 0.5 mL of acetic acid (13 M, [acetic acid]_{final}=4.0 M) at room temperature for 10 min (FIG. 4). The solution was cleaned using dialysis in H₂O to obtain a stable colloidal suspension.

Control experiment: TTA-DFP-nCOF synthesis in pure acetic acid. TTA-DFP-nCOF was synthesized by co-condensation of 2,6-diformylpyridine (DFP, 21 mg, 0.15 mmol, 5 equivalents) and 4,4′,4″-(1,3,5-triazine-2,4,6-triyl)trianiline (TTA, 12 mg, 0.03 mmol, 1 equivalent), in 3 mL of anhydrous 1,4-dioxane in presence of 0.5 mL of acetic acid (17 M, ([acetic acid]_{final}=5.0 M) at room temperature for 10 min (FIG. 4). The solution was cleaned using dialysis in H₂O to obtain a stable colloidal suspension.

Insulin loading in TTA-DFP-nCOF. Insulin was loaded into TTA-DFP-nCOF by a simple impregnation method. TTA-DFP-nCOF (5 mg) was suspended in 2 mL HEPES buffer, then a HEPES-buffered aqueous insulin solution ([insulin]=10 mg·mL⁻¹, 1 mL) was added (TTA-DFP-nCOF:Insulin ratio=1:2). The solution (pH 7.4) was stirred overnight at room temperature, cleaned with water several times by centrifugation and finally washed with deionized H₂O to remove unloaded insulin molecules.

Insulin-FITC loading in TTA-DFP-nCOF. Insulin-FITC (Sigma-Aldrich) was loaded into TTA-DFP-nCOF by a simple impregnation method. TTA-DFP-nCOF (5 mg) was suspended in 2 mL HEPES buffer, then a HEPES-buffered aqueous insulin solution ([insulin-FITC]=10 mg·mL⁻¹, 1 mL) was added (TTA-DFP-nCOF:Insulin-FITC ratio=1:2). The solution (pH 7.4) was stirred overnight at room temperature, cleaned with water several times by centrifugation and then washed with deionized H₂O to remove unloaded insulin-FITC molecules.

Insulin-loading in TTA-DFP-nCOF to achieve 30% loading capacity. Insulin was loaded into TTA-DFP-nCOF by a simple impregnation method. TTA-DFP-nCOF (5 mg) were suspended in 2 mL HEPES buffer, then a HEPE buffered aqueous insulin solution ([insulin]=2 mg·mL⁻¹, 0.2 mL) was added (TTA-DFP-nCOF:Insulin ratio=2:1). The solution (pH 7.4) was stirred overnight at room temperature, cleaned with water several times by centrifugation and finally washed with deionized H₂O to remove unloaded insulin molecules.

Glucose loading in TTA-DFP-nCOF. Glucose was loaded into TTA-DFP-nCOF by impregnation. TTA-DFP-nCOF (5 mg) were suspended in 2 mL HEPES buffer, then an aqueous glucose solution ([glucose]=5 mg·mL⁻¹, 1 mL) was added. The solution (pH 7.4) was stirred at room temperature overnight. The solution was then cleaned with water several times by centrifugation and washed with deionized H₂O to remove unloaded glucose molecules.

### Characterizations

High resolution transmission electron microscopy (HRTEM). High resolution transmission electron microscopy (HRTEM) images were obtained using a Talos F200X Scanning/Transmission Electron Microscope with a lattice-fringe resolution of 0.14 nm at an accelerating voltage of 200 kV equipped with a CETA 16M camera. The samples were prepared on holey carbon film mounted on a copper grid. A drop of diluted particle solution was spotted on the grid and dried overnight at room temperature (298 K). The obtained images of periodic structures were analyzed using TIA software. All the relevant areas were marked using bright field imaging mode at spot size 3 and the marked areas were also scanned using the STEM-HDAAF mode at spot size 9 for imaging and spot size 6 for conducting the STEM-EDAX. The STEM mode helps in providing the elemental composition as it works on the principle of mass determination. Such measurements can be performed at low electron dose by collecting the high-angle dark-field signal using an annular detector. This mode is generally used to image the elements that have different masses, with the heavier mass element appearing brighter. The samples were scanned at spot size 9 and with screen current of 60 pA. The data was analyzed using Velox analytical software.

Elemental Mapping. The chemical mapping was carried out in STEM-EDAX mode wherein the energy-dispersive X-ray analysis (EDAX) was carried out using a super-X EDS detector. The system has superior sensitivity with resolution of ≤136 eV@Mn-Kα for 10 kcps at zero-degree sample tilt. The detector provides quick data even for low intensity EDS signals. The data is the sum of 4 detectors and the collection time for the elemental maps in fast mapping mode can be reduced to minutes from hrs. The data was analyzed using Velox analytical software. The samples for the HRTEM study were prepared on holey carbon film mounted on a copper grid.

AFM Analysis. See FIG. 13.

Dynamic light scattering (DLS) characterization. DLS measurements were carried out on a Zetasizer Nano-ZS (Malvern Instruments) to determine the Zeta(ζ)-potential as well as the hydrodynamic size of the nanoparticles. All samples were analyzed at room temperature in 100 mM HEPES.

Powder X-ray diffraction (PXRD) measurements. Powder X-ray diffraction (PXRD) measurements were carried out to confirm the crystalline nature of the framework. The TTA-DFP-nCOFs were found highly crystalline in nature. In fact, we observed a strong peak at 2θ of 4.9° assigned to the (110) plane of the regularly ordered lattice. TTA-DFP-nCOF shows a broad peak at ˜24.80 corresponding to the reflection from the (003) plane.

N₂ adsorption-desorption experiments. N₂ adsorption-desorption isotherms were obtained at 77 K using Micrometrics ASAP 2020 surface area analyzer. Specific surface areas (S_{BET}) of the samples were calculated using Brunaur-Emmet-Teller (BET), whereas the pore volume (Vₚ) and pore size distribution (D_{BJH}) curves were obtained from Barrett-Joyner-Halenda (BJH) method.

Before measurements, the TTA-DFP-nCOF (empty or loaded with Insulin) was activated at 358 K for 24 h to remove the solvent and trapped gas. Based on the IUPAC classification system, TTA-DFP-nCOF exhibited type-II isotherms, which are indicative of microporous materials. BET surface area was found to be 384.52 m² g⁻¹.

Fourier Transform infrared (FTIR) spectroscopy. The TTA-DFP-nCOF formation as well as insulin loading was confirmed and characterized by ATR-IR spectroscopy using an Agilent Technologies Cary 600 Series FTIR spectrometer. The spectral data within the range of 4000 to 600 cm⁻¹ were recorded, and 512 scans were averaged for each spectrum with a spectral resolution of 2 cm⁻¹. The spectrum of the background was recorded first and it was subtracted from the spectra of samples automatically.

TTA-DFP-nCOF characterization in stomach conditions (pH=2). See FIG. 20.

¹H NMR spectroscopy. 1H NMR spectroscopy was used to observe the uptake of insulin by TTA-DFP-nCOF. Determination of the amount of insulin-FITC loaded in TTA-DFP-nCOF using fluorescence spectroscopy. At 37° C., pH 7.4 and λₑₓ=488 nm, TTA-DFP-nCOF are not intrinsically fluorescent. This indicates that fluorescence quenching occurs within the TTA-DFP-nCOF construct. Such quenching can be attributed to electronic interactions between the excited Insulin-FITC and the TTA-DFP-nCOF, or to self-quenching of the dye in the nanoparticles where the effective concentration of the protein is relatively high. The intensity of solution fluorescence was measured in comparison to a calibration curve (FIG. 22).

To estimate the amount of Insulin-FITC loading into TTA-DFP-nCOF, the unloaded insulin-FITC in the supernatant was determined by fluorescence spectroscopy based on comparison to a calibration curve of insulin standard solution.

DLS and Zeta(ζ)-potential characterization of TTA-DFP-nCOF/insulin. See FIGS. 25 and 26.

X-ray photoelectron (XPS) spectroscopy. X-ray photoelectron spectroscopy (XPS) analysis was performed in order to analyse the elemental composition of TTA-DFP-nCOF before and after addition of insulin, as well as to understand the interactions occurring between the nCOF surface and the protein. XPS experiments were carried out on a Kratos Axis Ultra DLD spectrometer under a base pressure of ˜ 2×10⁻¹⁰ mbar. A monochromated Al Kα X-ray source (1486.69 eV) was used to irradiate samples at room temperature. XPS spectra were recorded from an analysis area of 700 μm×300 μm. High-resolution XPS data of core levels was obtained with an energy resolution of 0.05 eV. For consistency, XPS measurements were calibrated to C1s (˜ 285 eV). Data was analyzed using CasaXPS package with Shirley background subtraction.

pH- and glucose dependent Insulin-FITC Release from TTA-DFP-nCOF by fluorescence emission spectroscopy.

Insulin release conditions. The effect of pH on the release of Insulin-FITC from TTA-DFP-nCOF was monitored over time in water buffered with PBS (10 mM) at 37° C. and pH=2.0 and 7.4. The pH of the solutions was adjusted using a 1 M HCl(aq) solution.

The effect of the glucose concentration on the release of insulin-FITC from TTA-DFP-nCOF was monitored using fluorescence spectroscopy over time in water buffered with PBS (10 mM) at 37° C. and in several glucose concentrations ([glucose]=0, 1 3, and 5 mg·mL⁻¹).

The effect of human serum, a mix of 11 amino acids (alanine, arginine, asparagine, aspartate, cysteine, glutamate, glutamine, glycine, proline, serine, tyrosine), a saline solution of fructose (3 mg mL⁻¹) or sucrose (3 mg mL⁻¹) on the release of insulin-FITC from TTA-DFP-nCOF was monitored over time in water buffered with PBS (10 mM) at 37° C. and in hyperglycemic conditions ([glucose]=3 mg·mL⁻¹).

At regular intervals, samples were withdrawn and the fluorescence intensity was measured.

Circular dichroism (CD) spectroscopy. To evaluate the changes of the activity and structure of insulin released from the nanoparticles, circular dichroism (CD) spectroscopy was performed as a common method to analyze the secondary structure of a protein with high reliability. In the CD spectra of the native insulin in HEPES (pH 7.4), there were two extrema at 208 and 222 nm, due to the α-helix structure and β-structure, respectively.

The chemical stability of insulin loaded in TTA-DFP-nCOF exposed to the GI fluid simulations (pH 2.0, 24 hours) was evaluated. At acidic pH, insulin is not released from the nanoparticle, therefore in order to perform CD analysis we exposed TTA-DFP-nCOF/insulin to NaOH (0.1 M) to release the protein from the NPs. As presented in FIG. 32, far-UV CD spectroscopy of the insulin released from NPs and pure insulin solution showed two negative bands at 208 nm and 222 nm which correspond to the predominant α-helix structure and β-pleated sheet structure, respectively. The ratio of intensity of 208 and 223 nm bands ([Φ]208/[Φ]223) has usually been employed to provide a qualitative measure of insulin association. The [Φ]208/[Φ]223 ratios of native and GI fluid exposed insulin from TTA-DFP-nCOF were both 1.2 which reflected that there was no significant difference in the secondary structure between the native and GI fluid exposed TTA-DFP-nCOF/insulin.

The CD spectrum of the insulin after releasing for 12 hours from the TTA-DFP-nCOF/insulin under the hyperglycemic environment was similar to that of the native insulin with two extrema (FIG. 33). The ratios between bands ([φ]208/[φ]222) for the native and released insulin were 1.25 and 1.24, respectively. Therefore, the secondary structure of the insulin released from the nanoparticles was similar to the original insulin. Accordingly, the released insulin maintained its structure and properties.

Glucose interaction. In order to study the release mechanism of insulin triggered by glucose, we incubated the TTA-DFP-nCOF 24 hours and 37° C. with i) insulin alone, ii) glucose alone (5 mg·mL⁻¹) and iii) insulin followed by 24 hours with glucose (5 mg·mL⁻¹). Samples were washed thoroughly and freeze-dried using lyophilization. Loading efficiency (wt %) was calculated using mass differences by comparing with the TTA-DFP-nCOF mass.

### In Vitro Biological Studies.

Cell culture. Hepatocellular carcinoma (Hep-G2, ATCC HB-8065), colorectal carcinoma (HCT-116, ATCC CCL-247), colon carcinoma (RKO, ATCC CRL-2577), cervical adenocarcinoma (Hela, ATCC CCL-2), breast adenocarcinoma (MCF-7, ATCC HTB-22), metastatic breast adenocarcinoma (MDAMB-231, ATCC HTB. 26), epithelial embryonic kidney (HEK293-T, ATCC CRL-3216) and malignant glioblastoma (U251-MG, ATCC 09063001) human cell lines were cultured in Dulbecco's Modified Eagle's medium (DMEM) supplemented with 10% fetal bovine serum (FBS), 1% penicillin/streptomycin and 20 mL L-glutamine at 5% CO₂ and 37° C.

Ovarian cancer (A2780, ECACC 93112519) and intestine ileocecal adenocarcinoma (HCT-8 ATCC CCL-244) human cell lines were cultured at 5% CO₂ and 37° C. in Roswell Park Memorial Institute (RPMI)-1640 medium complemented with 10% fetal bovine serum (FBS) and 1% penicillin/streptomycin.

In vitro cell viability. Cell viability was assessed using CellTiter-Blue® Cell Viability assay (CTB, Promega). The assay measures the metabolic reduction of a non-fluorescent compound, resazurin, into a fluorescent product, resofurin, in living cells. As non-viable cells rapidly lose their metabolic activity, the amount of the resofurin product can be used to estimate the number of viable cells following treatment. Once produced, resofurin is released from living cells into the surrounding medium. Thus, the fluorescence intensity of the medium is proportional to the number of viable cells present.

96-well plates were seeded with Hep-G2cells (˜5,000 cells per well in 100 μL of DMEM) and incubated at 37° C. for 24 hours. The medium was removed and replaced with fresh medium (control) or various concentrations of test compounds and incubated at 37° C. for 48 hours. Thereafter, cells were incubated with 80 μL DMEM and 20 μL of CTB per well for 6 hours at 37° C. The fluorescence of the resofurin product (λ_{ex/em} 560/620) was measured. Untreated wells were used as control.

The percentage of cell viability were calculated using the following formula:

$$Viability ( \% ) = [ \{ {F}_{t r e a t e d} - {F}_{blank} ) / ( {F}_{c ontrol} - {F}_{blank} ) ] \times 100$$

All assays were conducted in triplicate and the mean IC₅₀±standard deviation was determined.

Intracellular distribution of nanoparticles using TEM analysis. TEM was used to investigate the fate of TTA-DFP-nCOF/insulin in cells, their impact on cellular structures and their interactions with organelles on 2 colon cell lines (RKO and HCT-116, 4 hours incubation times) and analyzed 4 h, 24 h and 48 h post-treatment.

For TEM analysis, cells were seeded in T75 flasks in complete DMEM and incubated 4 hours with cell-medium alone (control), TTA-DFP-nCOF/insulin ([TTA-DFP-nCOF]=50 μg·mL⁻¹ in DMEM). Cells were harvested 4, 24 and 48 hour post-treatment. Cell pellets were washed twice with phosphate-buffered saline (PBS). The cells were cryo-fixed within a few milliseconds at a pressure of 2000 bar under liquid nitrogen using a high-pressure freezer (Leica Microsystems, Germany). After freezing, the sample pod was released automatically into a liquid nitrogen bath. While still in liquid nitrogen, the sample carrier was separated from the specimen pod using precooled fine-tipped tweezers and transferred to the cryo-transfer storage box for the flat specimen carrier, where the samples were stored in preparation for freeze substitution. Freeze substitution was performed using an automatic freeze substitution (AFS) unit (Leica EM AFS2, Heerbrugg, Switzerland) in a 10 mL solution of cold dry absolute acetone (v/v) containing 1% osmium tetroxide (w/v), 0.5% uranyl acetate (w/v) and 5% distilled water (v/v). The AFS unit was slowly warmed from −90° C. to 0° C. (2° C./h), with the temperature being held at both −60° C. and −30° C. for a period of 8 h. Samples were transferred to room temperature in a closed container to prevent condensation, rinsed with absolute acetone (3×5 minutes) and infiltrated with 30, 60 and 100% Epon resin for 3 h each. Epon was exchanged and individual samples were embedded in 1 mL Eppendorf® lids for 24 h at 60° C. Finally, the samples were sectioned with an cryo ultra-microtome (Leica UC7/FC7) at room temperature using a diamond knife, and the ultrathin sections were examined under TEM (Talos F200X STEM).

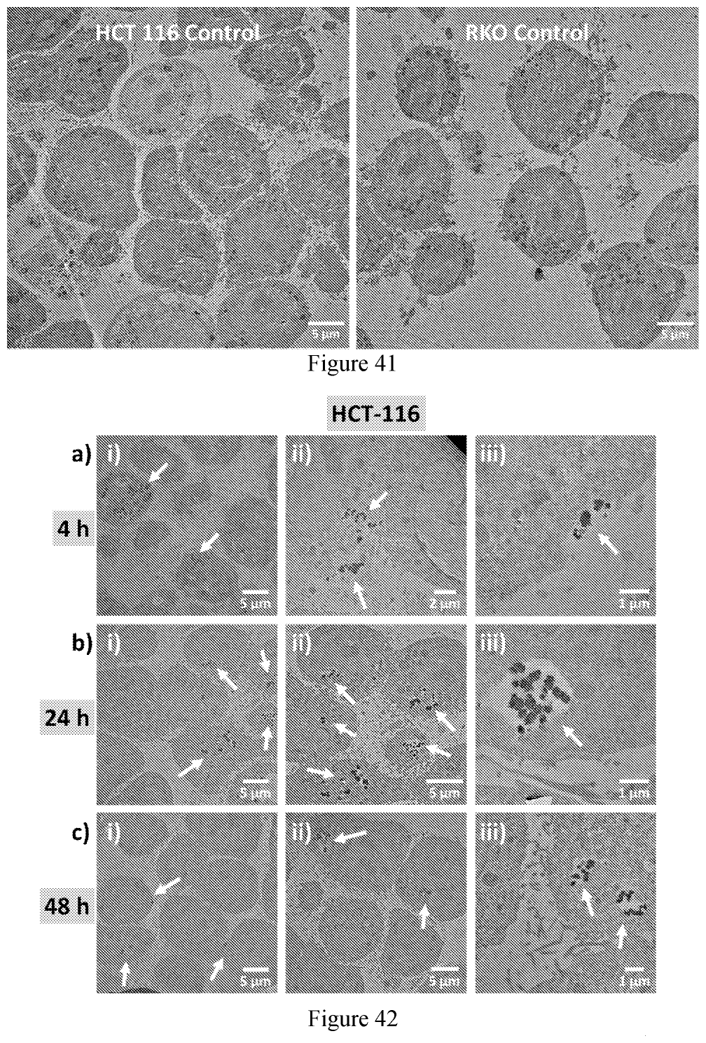

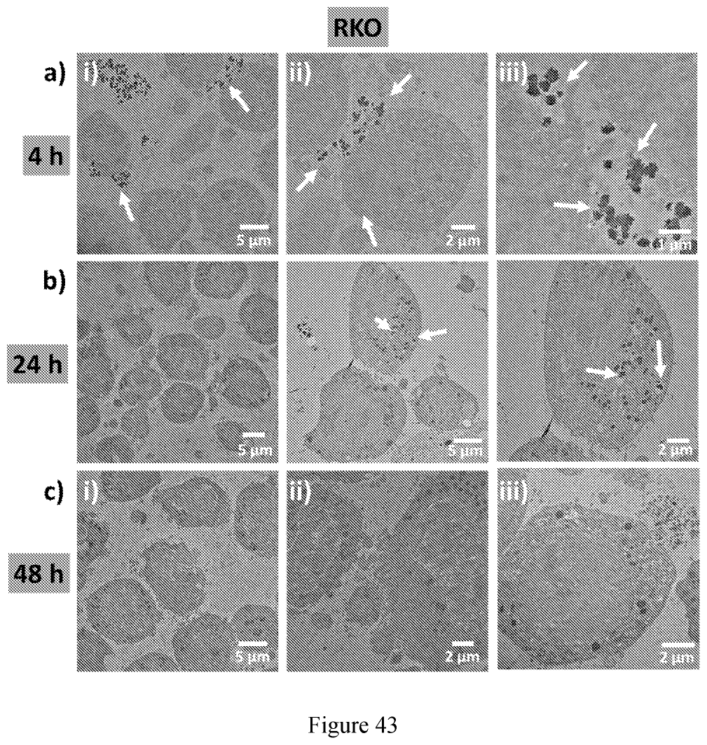

The TEM images of untreated HCT-116 and RKO cells (FIGS. 42-43) showed typical morphological features of cells, including intact cellular membranes; numerous microvilli and membrane blebbing on the surface of plasma membranes; a well-developed rough endoplasmic reticulum, a large Golgi apparatus and other organelles or structures, such as mitochondria, small vacuoles and granules; centric large nuclei with obvious nucleoli, surrounded by intact nuclear membranes; and, a normal distribution of heterochromatin.

All TTA-DFP-nCOF/insulin treated samples showed regular ultrastructure of the RKO and HCT-116 cells, with a roundish cellular shape and a plasma membrane rich in protrusions (such as microvilli), a well-developed rough endoplasmic reticulum, Golgi apparatus, and mitochondria, which indicate the maintenance of metabolic active cells (FIGS. 42-43).

After 4 h of incubation, significant amounts of TTA-DFP-nCOF/insulin can be visualized within some of the treated cells and at their surface (FIGS. 42-43). TTA-DFP-nCOF/insulin undergo endocytosis and are located in endosomes. Membrane deformation was also observed, confirming the internalization of TTA-DFP-nCOF/insulin by endocytosis. The TTA-DFP-nCOF/insulin which are internalized within the cell vacuoles could mainly be found as aggregates. Furthermore, we did not find any TTA-DFP-nCOF/insulin near the nucleus, as it is plausible these aggregates would be physically unable to breach the nuclear membrane pores with sizes in the range of 10-20 nm. After 24 h, TTA-DFP-nCOF/insulin could be located inside both cell lines vacuoles in the perinuclear region but no more on the membrane; cells continue growing with cells dividing (FIGS. 42-43). After 48 h, some HCT-116 cells still contain nanoparticles inside vacuole in the cytoplasm but most cells do not; cells continued growing and multiplying (FIG. 42). In RKO cells, no nanoparticles could be detected (FIG. 43).

Hemolysis assay. When the external membrane of the erythrocytes is destroyed, hemoglobin is released. It is possible to estimate the amount of destroyed erythrocytes in a given test by measuring the quantity of hemoglobin in a sample by spectrophotometry. Human blood was obtained from 3 healthy donors. 2.0 mL of an ethylenediaminetetraacetate-stabilized blood sample was added into 4 mL of physiological saline and then red blood cells were isolated by centrifugation (3000 rpm, 8 min). The red blood cells were washed five times with physiological saline buffer (PBS) and diluted into 2% red blood cell suspension. TTA-DFP-nCOF or TTA-DFP-nCOF/insulin (0.75, 1.5 and 3.0 mg·mL⁻¹) was added into the red blood cell suspensions at the predetermined concentration and mixed using a gentle vortex. Meanwhile, physiological saline with or without Triton X-100 (0.3%) was added into the red blood cell suspensions as negative and positive controls, respectively. Samples were placed in a static condition at 37° C. for 1 h. Finally, all samples were centrifuged at 5000 rpm and 100 μL of the supernatant was placed into a 96-well plate for detection at the wavelength of 540 nm. The hemolysis ratio (HR) represents the degree of red blood cell membranes destroyed in the samples.

$$HR ( \% ) = \frac{{A}_{sample} - {A}_{negative control}}{{A}_{positive control} - {A}_{negative control}} \times 100$$

Aₛₐₘₚₗₑ, Aₚₒₛᵢₜᵢᵥₑ control, and A_{negative control} represented the absorbance of the sample, the positive control, and the negative control, respectively. These tests were performed in triplicate.

Ex Vivo permeation study across mouse intestinal sac. Ex vivo absorption evaluation was carried out by permeation measurements in excised rat small intestine as described elsewhere. Mice (25 g) were anaesthetized with isoflurane, killed and exsanguinated. As the animal was placed under anesthesia and euthanized for a different purpose, no ethical approval was necessary for retrieval of the tissue. Freshly excised intestinal tissues were washed with PBS and cut into pieces of 5-4 cm. 0.3 mL of TTA-DFP-nCOF/insulin-FITC (1 mg·mL⁻¹) was syringed into intestinal sacs; the filled tissues were incubated in oxygenated tissue culture Dulbecco's Modified Eagle's Medium (DMEM, 10 mL) at 37° C. Sample solution (0.1 mL) was withdrawn from the serosal side at fixed time intervals up to 180 min and replaced with fresh medium. Fluorescence signal for FITC was measured on a fluorescent plate reader, (FITC excitation/emission: 495 nm/519 nm) and compared to a standard curve of log dilutions for TTA-DFP-nCOF/insulin-FITC ranging from 1 to 1×10⁻⁶ M. Tests were carried out in triplicate on three different intestinal segments from three different mice.

$$Apparent permeability ( μg \cdot {cm}^{- 2} ) = \frac{Concentration \times volume}{Mucosal surface area}$$

To calculate mucosal surface area, the intestine was considered as a cylinder and the following equation was used:

$$\begin{matrix}Mucosal surface area ( {cm}^{- 2} ) = 2 π r ( h + r ) \\ where h = length and r = radius of intestinal sac\end{matrix}$$

At the end of the experiments, tissues were washed with normal saline, and drug accumulation in the gut wall was investigated by TEM of the serosal medium as well the intestinal tissue.

Intestinal tissues were fixed in 4.5% paraformaldehyde solution. The fixed samples were cryo-fixed within a few milliseconds at a pressure of 2000 bar under liquid nitrogen using a high-pressure freezer (Leica Microsystems, Germany). Freeze substitution was performed using an automatic freeze substitution (AFS) unit (Leica EM AFS2, Heerbrugg, Switzerland) in a 10 mL solution of cold, dry, absolute acetone (v/v) containing 1% osmium tetroxide (w/v), 0.5% uranyl acetate (w/v), 5% distilled water (v/v) and embedded with epoxy resin. Subsequently, an ultrathin intestinal specimen was made and observed with high-resolution TEM (Talos F200X STEM).

In vivo animal assessments. All animals were raised in accordance with the policies of the University of Tlemcen Institutional Animal Care and Use Committee (IACUC) (accreditation number: D01N01UN130120150006).

Animals. Wistar rats (12 weeks, 200 g±20) were used for this study. They were obtained from Pasteur Institute (Algiers, Algeria). Rats were housed individually in wood-chip bedded plastic cages at constant temperature (25° C.), maintained on a 12:12 hours light/dark cycle and fed with a standard pellet diet and water ad libitum. The study was conducted in accordance with the national guidelines for the care and use of laboratory animals.

TID Induction. Type 1 Diabetes was induced via a single intraperitoneal injection of streptozotocin (STZ, dissolved in 10 mM citrate buffer at pH 4.5) at the STZ dose of 45 mg·kg⁻¹ body weight. Rats were returned to their cages, and given food and water for the next 4 days till the induction of diabetes. The blood glucose level was monitored using a blood glucose monitoring system (AccuChek Performa, Hoffman-La Roche) by taking samples from a rat tail vein. The rats showing fasting blood glucose level ≥250 mg/dL (13.7 mmol·L⁻¹) were considered as diabetic and were selected for the studies. The rats were fasted overnight and remained fasted during the period of experiment, but were allowed to drink water.

In vivo hypoglycemic effect. Rats were randomly divided into six groups (n=3), the formulations administered to the TID rats were as follows: 1) TTA-DFP-nCOF/insulin administered by oral gavage (o.g., 50 IU·kg⁻¹); 2) TTA-DFP-nCOF administered by oral gavage (o.g., 2 mg·kg⁻¹); 3) insulin solution administered by oral gavage (o.g., 50 IU·kg⁻¹); 4) insulin solution administered subcutaneously (5 IU·kg⁻¹) set as the positive control with 100% pharmacological availability of insulin; 4) untreated diabetic rats and 6) non-diabetic rats. Blood glucose level was determined with a glucometer. Blood samples were taken from the tail veins every hour for 10 hours.

In vivo plasma insulin level. Rats were randomly divided into 3 groups (n=3), the formulations administered to the TID rats were as follows: 1) TTA-DFP-nCOF/insulin administered by oral gavage (o.g., 50 IU·kg⁻¹); 2) insulin solution administered subcutaneously (SC, 5 IU·kg⁻¹) set as the positive control with 100% pharmacological availability of insulin and 3) untreated diabetic rats. Pharmacological effect was determined by measuring the increase of serum insulin in diabetic rats during 10 h. Insulin plasma level was evaluated using a Rat Ins1 Insulin ELISA Kit from Sigma-Aldritch (RAB00904-1KT). Blood samples were collected from the tail veins every hour over 10 hours. The area above the curve (AAC) was calculated using the trapezoidal method. The pharmacological availability (PA) calculated as the cumulative hypoglycemic effect relative to 100% PA of sc. free insulin was determined using the equation:

$$Pharmacological Availability ( PA ) = \frac{AAC oral TTA ‐ DFP ‐ nCOF / Insulin \times Dose insulin sc .}{AAC insulin sc . \times Dose oral TTA ‐ DFP ‐ nCOF / Insulin}$$

Homeostatic model assessment (HOMA) of insulin resistance (HOMA-IR) and insulin-sensibility (HOMA-IS) of β-cell function were calculated using the following equations:

$$\begin{matrix}HOMA ‐ IR = \frac{Fasting glucose [ mg . {dL}^{- 1} ] \times Fasting insulin [ mU . {L}^{- 1} ]}{405} \\ HOMA ‐ IS = \frac{10000}{Fasting glucose [ mg . {dL}^{- 1} ] \times Fasting insulin [ m U . {L}^{- 1} ]}\end{matrix}$$

In vivo oral glucose tolerance test (OGTT). For oral glucose tolerance test (OGTT), rats were randomly divided into 3 groups (n=3) corresponding to i) TTA-DFP-nCOF/insulin administered by oral gavage (o.g., 50 IU·kg⁻¹); ii) insulin solution administered subcutaneously (SC, 5 IU·kg⁻¹) and iii) untreated diabetic rats. Animals have first received the TTA-DFP-nCOF/insulin by oral gavage or the subcutaneous insulin injection. 3 hours after that, they received 2.5 g·kg⁻¹ of glucose dissolved in 1 ml of water and glycaemia was evaluated for 280 min. Glycaemia was measured at time zero (basal) and every 30 min up to 280 min after gavage of 2.5 g·kg⁻¹ body weight of glucose from the tail vein.

Histopathology. The collected tissues were embedded in paraffin after fixing the tissues in 10% formalin. Serial sections were cut and stained with hematoxylin and eosin. The sections were examined under high-power microscope (200×) and photomicrographs were taken.

SC insulin-treated rats' livers compared to the non-diabetic control displayed an increase of big hepatocytes, a necrosis of hepatocytes and a narrowing in the sinusoids (FIG. 3g-ii) due to the STZ-induced diabetes. Histopathological study of the livers of the group treated with TTA-DFP-nCOF/insulin showed similar structures to the non-diabetic rats, with normal hepatocytes and sinusoids. Regarding the kidney function, SC insulin-treated rats displayed an increase of the size of Bowman capsules, hypertrophy of the glomeruli and necrosis of the tubules (FIG. 3g-v) also due to STZ administration to induced diabetes. The kidneys of the rats treated with TTA-DFP-nCOF/insulin showed fewer alterations than the subcutaneous insulin rat kidneys, smaller Bowman's spaces and well-individualized tubules.

Biochemical determinations. Liver function test was carried out using serum biomarkers such as aspartate amino-transferase (AST) and Alanine transaminase (ALT) measured from the plasma obtained from the tail vein using SPINREACT kit. Kidney function test was performed using as urea and creatinine measured by SPINREACT kit.

### Example 2

This example describes use of COF nanoparticles of the present disclosure to treat diabetes type 2.

TTA-DFP-nCOF transports insulin into insulin-resistant cells to stimulate long-term glucose consumption, contrasting with the temporary effect of free insulin that binds to cell membranes. In comparison to the two FDA-approved technologies, our system is biocompatible, highly stable in the stomach, cost effective, specific and glucose-responsive, therefore represent a step forward in the future of Insulin oral delivery and a novel pathway toward the treatment of type 1 and 2 diabetes through nCOF-based insulin oral delivery.

Recent studies have shown that internalization of insulin into cells can overcome what is known as insulin resistance, a phenomenon whereby a change in physiological regulation results in fixed doses of insulin having no effect on glucose metabolism to the extent that it does in normal individuals. Intracellular delivery of insulin is a challenging approach due to the inefficiency of insulin in penetrating cells and the rapid degradation of insulin when trapped in the cytoplasm instead of being released inside cells. With insulin resistance a predictor of Type 2 diabetes and since the liver is one of the organs targeted by insulin in Type 2 diabetes, the human hepatoma cell line (Hep-G2) was used for in vitro assessment of nCOF-insulins effectiveness for internalization of insulin into the cell.

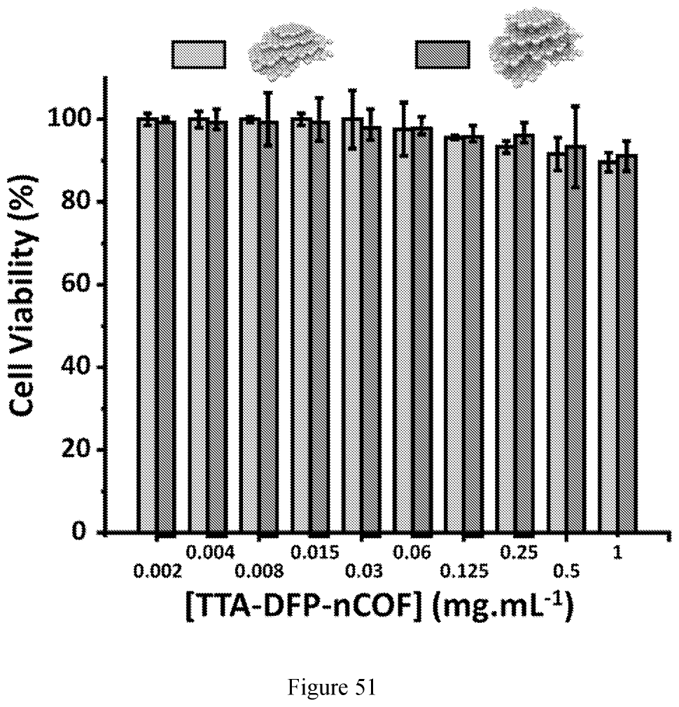

In vitro viability studies were carried out in Hep-G2 cells (FIG. 51) to demonstrate the potential of TTA-DFP-nCOF as a biocompatible delivery vehicle. Both TTA-DFP-nCOF and TTA-DFP-nCOF/Insulin elicited no cytotoxic effects at TTA-DFP-nCOF concentrations up to 1 mg mL⁻¹ following 48 h of incubation indicating desirable biocompatibility.

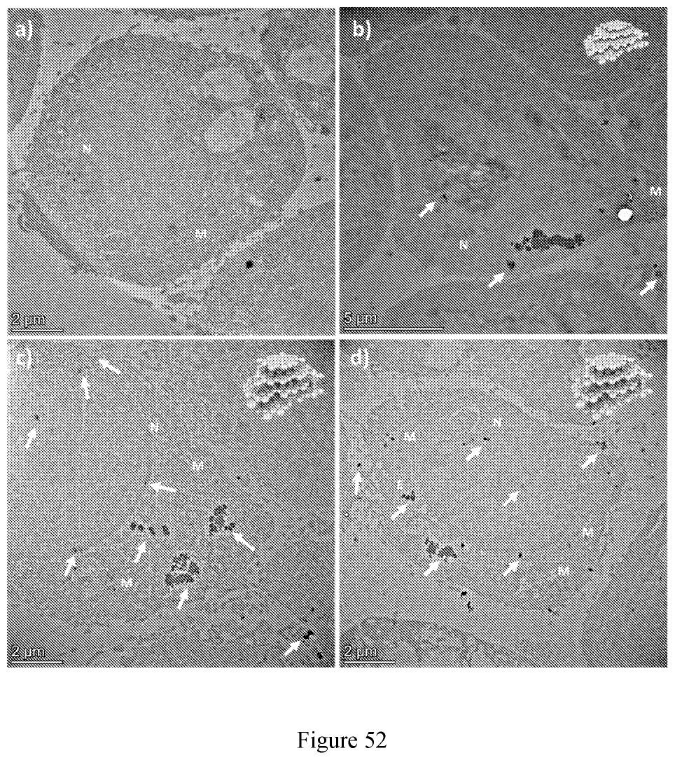

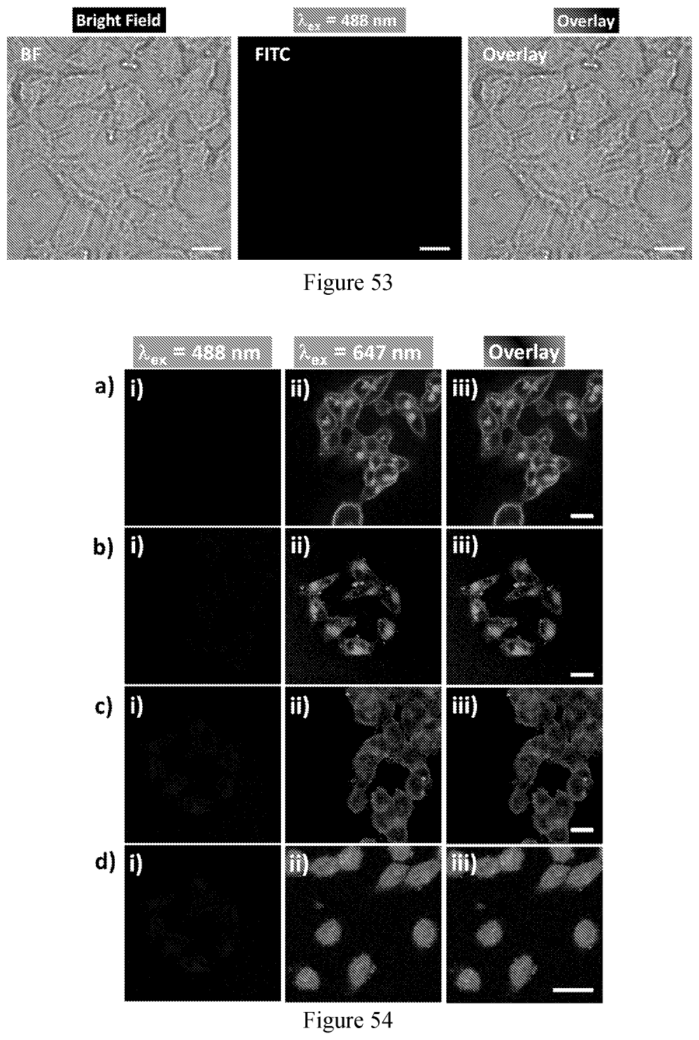

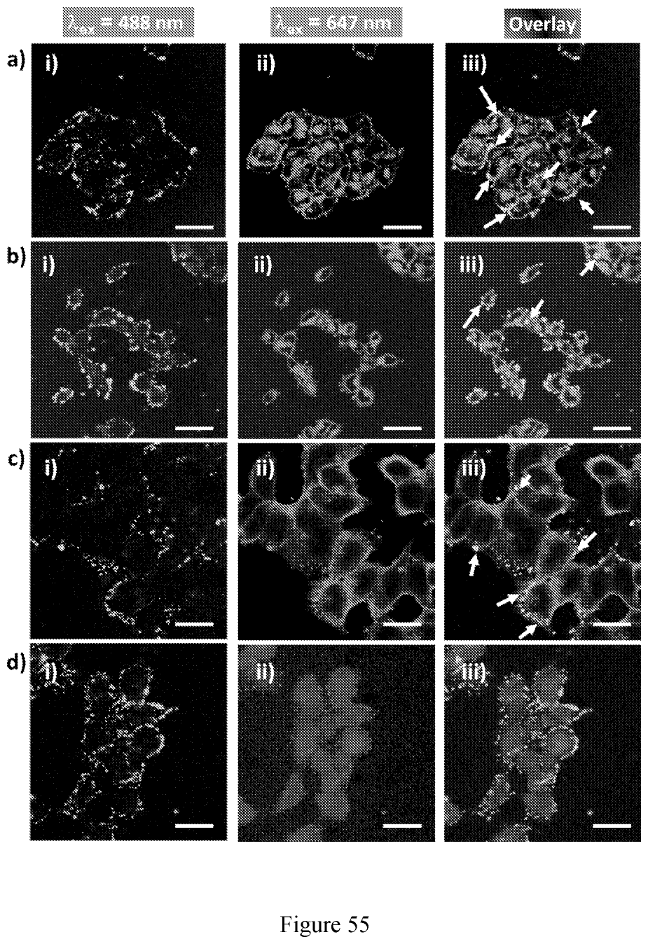

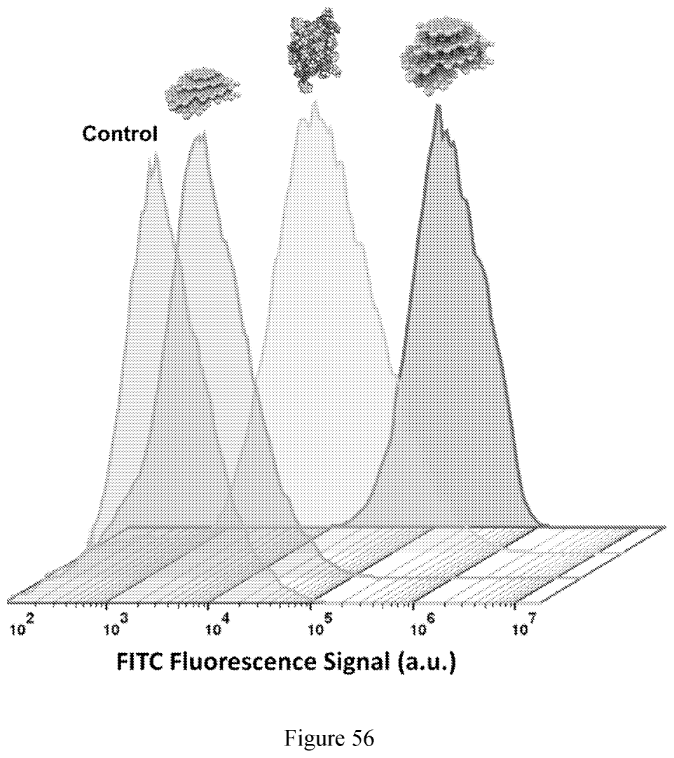

Localization of TTA-DFP-nCOF/Insulin inside Hep-G2 cells was investigated by TEM (FIG. 52). After 4 h of incubation, nanoparticles were primarily observed in the cytoplasm and the nucleus of the cells. Nanoparticles were detected transiting through the cells and excreted at the cell basolateral side, thus confirming biocompatibility. Using confocal microscopy (FIGS. 53-55) there was no detectable intracellular insulin-FITC signal in the insulin-treated cells, indicating that free insulin in its molecular form was not able to enter cells, while co-localization of the insulin-FITC signal with cellular organelle markers of cells treated with TTA-DFP-nCOF/Insulin-FITC showed that after 4 h of incubation insulin-FITC is bound to the membrane, inside the lysosome as well as in the cytoplasm but not in the nucleus. TTA-DFP-nCOF/Insulin-FITC were internalized in the cytoplasm of the cells, triggering the release of insulin, whereas free insulin merely entered the cells (FIG. 55). Flow cytometry showed a 15-fold increase in fluorescence in cells treated with TTA-DFP-nCOF/Insulin-FITC as compared to those treated with the free insulin (FIG. 56).

Resistant-HepG2 (R-HepG2) cells were obtained using an established method and their glucose metabolism was measured to be 58% of normal HepG2 cells (control, defined as 100% glucose metabolism) (FIG. 1). Three treatments were administered; free-form insulin, TTA-DFP-nCOF, and TTA-DFP-nCOF/Insulin, with glucose metabolism of treated R-HepG2 recorded at t=4 h and 24 h. Free-form insulin treatment resulted in a transient increase in cellular glucose metabolism of R-HepG2 after 4 h with a statistically significant worsening of the cells' insulin resistance after 24 h. This phenomenon was consistent with the short-term effect of insulin on diabetes. TTA-DFP-nCOF-treated cells were inert in terms of any glucose metabolism regulations. However, TTA-DFP-nCOF/Insulin treatment not only resulted in an improvement in glucose metabolism after 4 h (72%), this upregulation effect enhanced after 24 h, with glucose metabolism of TTA-DFP-nCOF-treated R-HepG2 cells almost reverting to that of normal HepG2. Thus, the observed long-term improvement should be attributed to the intracellular insulin delivered by TTA-DFP-nCOF.

TTA-DFP-nCOF to insulin resistant-liver cell demonstrate their potential in glucose upregulation and the disappearance of insulin resistance symptoms.

### In Vitro Biological Studies.

Cell culture. Human hepatocellular carcinoma (Hep-G2, ATCC No. HB-8065) cell line were cultured in Dulbecco's Modified Eagle's medium (DMEM) supplemented with 10% fetal bovine serum (FBS), 1% penicillin/streptomycin and 20 mL L-glutamine at 5% CO₂ and 37° C.

In vitro cell viability on Hep-G2 cells. Cell viability was assessed using CellTiter-Blue® Cell Viability assay (CTB, Promega). The assay measures the metabolic reduction of a non-fluorescent compound, resazurin, into a fluorescent product, resofurin, in living cells. As non-viable cells rapidly lose their metabolic activity, the amount of the resofurin product can be used to estimate the number of viable cells following treatment. Once produced, resofurin is released from living cells into the surrounding medium. Thus, the fluorescence intensity of the medium is proportional to the number of viable cells present.

96-well plates were seeded with Hep-G2cells (˜5,000 cells per well in 100 μL of DMEM) and incubated at 37° C. for 24 hours. The medium was removed and replaced with fresh medium (control) or various concentrations of test compounds and incubated at 37° C. for 48 hours. Thereafter, cells were incubated with 80 μL DMEM and 20 μL of CTB per well for 6 hours at 37° C. The fluorescence of the resofurin product (λ_{ex/em} 560/620) was measured. Untreated wells were used as control.

The percentage of cell viability were calculated using the following formula:

$$Viability ( \% ) = [ ( {F}_{t r e a t e d} - {F}_{blank} ) / ( {F}_{control} - {F}_{blank} ) ] \times 100$$

All assays were conducted in triplicate and the mean IC₅₀±standard deviation was determined.

Intracellular distribution of nanoparticles using TEM analysis. For TEM analysis, Hep-G2 cells were seeded in T75 flasks in complete DMEM and incubated 4 hours with cell-medium alone (control), TTA-DFP-nCOF or TTA-DFP-nCOF/Insulin ([TTA-DFP-nCOF]=0.5 mg·mL⁻¹ in DMEM). After harvesting, cell pellets were washed twice with phosphate-buffered saline (PBS). The cells were cryo-fixed within a few milliseconds at a pressure of 2000 bar under liquid nitrogen using a high-pressure freezer (Leica Microsystems, Germany). After freezing, the sample pod was released automatically into a liquid nitrogen bath. While still in liquid nitrogen, the sample carrier was separated from the specimen pod using precooled fine-tipped tweezers and transferred to the cryo-transfer storage box for the flat specimen carrier, where the samples were stored in preparation for freeze substitution. Freeze substitution was performed using an automatic freeze substitution (AFS) unit (Leica EM AFS2, Heerbrugg, Switzerland) in a 10 mL solution of cold dry absolute acetone (v/v) containing 1% osmium tetroxide (w/v), 0.5% uranyl acetate (w/v) and 5% distilled water (v/v). The AFS unit was slowly warmed from −90° C. to 0° C. (2° C./h), with the temperature being held at both −60° C. and −30° C. for a period of 8 h. Samples were transferred to room temperature in a closed container to prevent condensation, rinsed with absolute acetone (3×5 minutes) and infiltrated with 30, 60 and 100% Epon resin for 3 h each. Epon was exchanged and individual samples were embedded in 1 mL Eppendorf® lids for 24 h at 60° C. Finally, the samples were sectioned with an ultra-microtome at room temperature using a diamond knife, and the ultrathin sections were examined under TEM (Talos F200X STEM).

In vitro Insulin-FITC internalization in cells study by confocal microscopy. Hep-G2 cells were seeded on sterile cover-slips in complete DMEM and incubated for 24 h. Hep-G2 cells were incubated 4 hours with Insulin-FITC ([Insulin-FITC]=10 μM). The cells were stained with organelles markers to understand the internalization of TTA-DFP-nCOF/Insulin-FITC. Cells were incubated for 30 min either with NucRed® Live 647 ReadyProbes® Reagent (labelling nucleus), LysoTracker™ 647 Deep Red (labelling lysosomes), ActinRed™ 647 ReadyProbes™ Reagent (labeling cytoplasm) and CellMask™ 647 Deep Red (labeling membrane), followed by three cycles of PBS washing. Then, for each experiment, the cells were fixed with formaldehyde solution (3.7%) for 10 min followed by washing thrice with PBS. The cells were kept for 5 min in the PBS during washing cycles. ProLong Live Antifade Reagent was added to suppress photo-bleaching and preserves the fluorescent signals. The cover-slips was then fixed onto a microscope slide. The intracellular internalization of TTA-DFP-nCOF/Insulin-FITC was observed using confocal microscopy (Olympus FV1000MPE) measuring the fluorescence signal of the Insulin-FITC (488 nm) in Hep-G2 cells as well as the fluorescence emission from the 4 organelle markers labeling the plasmic membrane, the lysosomes, the cytoplasm as well as the nucleus (with excitation/emission of 647/668 nm).

Flow cytometry analysis. Hep-G2 cells were grown in petri dishes to a density of 100,000 cells/mL in DMEM for 24 h. Hep-G2 cells were incubated 4 hours with no additives (control), TTA-DFP-nCOF, TTA-DFP-nCOF/Insulin-FITC ([Insulin-FITC]=10 μM). Insulin-FITC uptake was measured with a BD Accuri C6 flow cytometer.

Insulin resistance. Insulin resistance is defined as a change in physiological regulation such that a fixed dose of insulin does not affect glucose metabolism to the extent that it does in normal individuals. R-HepG2 was obtained using an established method, and its glucose metabolism was measured to be 58% of normal HepG2; normal HepG2 was used as a control, and its glucose metabolism was defined as 100%.

Insulin resistant cells. HepG2 cells were plated in six-well plates at a density of 100 000 cells per well in growth medium. After 24 hours, the medium was changed to complete DMEM with 10⁻⁶ M dexamethasone (Alfa Aesar, USA) for another incubation period of 60 h to establish R-HepG2.

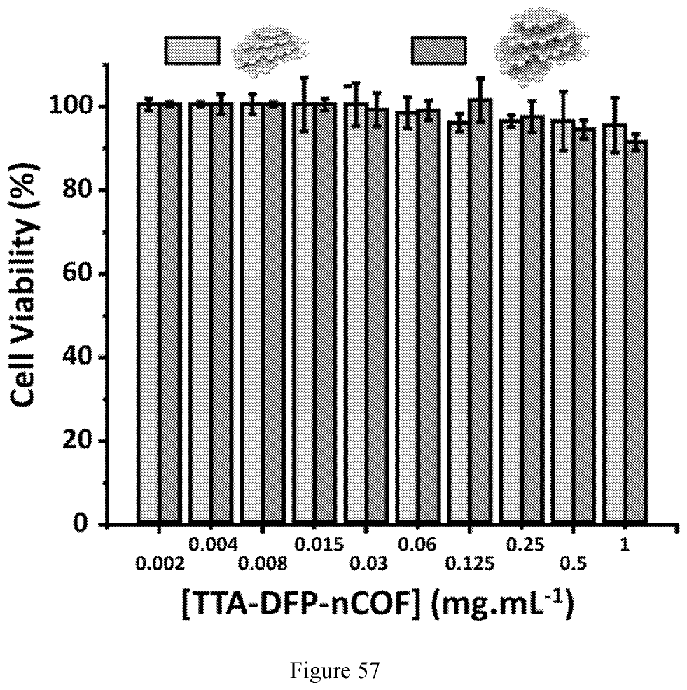

In Vitro Cell Viability on R-Hep-G2 Cells. See FIG. 57.

Glucose consumption assay. R-Hep-G2 cells were grown in petri dishes (100 000 cells/mL) in glucose- and phenol free DMEM DMEM for 24 h. R-Hep-G2 cells were incubated 4 hours at 37° C. with no additive, Insulin or TTA-DFP-nCOF/Insulin ([Insulin]=10 μM). The glucose residues in the cell culture media were measured at 4 and 24 hour-post-treatment using Amplex® Red Glucose/Glucose Oxidase Assay Kit (Invitrogen) as per vendor instructions.

Although the present disclosure has been described with respect to one or more particular embodiments and/or examples, it will be understood that other embodiments and/or examples of the present disclosure may be made without departing from the scope of the present disclosure.

1. A covalent organic framework (COF) nanoparticle, comprising 10-25 COF nanosheets, wherein the COF nanosheets are stacked in a staggered configuration and each COF nanosheet is a co-condensate of 2,6-diformylpyridine (DFP) and 4,4′,4″-(1,3,5-triazine-2,4,6-triyl)trianiline (TTA) and the COF nanoparticle has a longest linear dimension of 75-300 nm.

2. The COF nanoparticle according to claim 1, wherein the molar ratio during condensation of DFP and TTA is 5:1 (DFP:TTA).

3. The COF nanoparticle according to claim 1, wherein the COF nanoparticle has a longest linear dimension of about 120 nm.

4. The COF nanoparticle according to claim 1, wherein the COF nanoparticle has 16-20 COF nanosheets.

5. The COF nanoparticle according to claim 4, wherein the COF nanoparticle has 18 COF nanosheets.

6. The COF nanoparticle according to claim 1, wherein the COF nanoparticle incorporates a plurality of cargo proteins.

7. The COF nanoparticle according to claim 6, wherein the cargo protein is insulin.

8. The COF nanoparticle according to claim 7, wherein the insulin is 30-75 wt %, relative to the weight of the COF nanoparticle.

9. A composition comprising a plurality of COF nanoparticles according to claim 1 and a pharmaceutically acceptable carrier.

10. A method for making covalent organic framework (COF) nanoparticles, comprising:
contacting 2,6-diformylpyridine (DFP), 4,4′,4″-(1,3,5-triazine-2,4,6-triyl)trianiline (TTA), and an acid in a solvent to form a reaction mixture, wherein the reaction mixture is held at room temperature, wherein after a period of time, the covalent organic framework (COF) nanoparticles are formed; and
impregnating the COF nanoparticles with one or more cargo proteins.

11. The method according to claim 10, further comprising purifying the reaction mixture prior to the impregnating, wherein the purifying comprises dialyzing the reaction mixture in water.

12. The method according to claim 10, wherein the one or more cargo proteins are insulin.

13. The method according to claim 12, wherein the weight ratio of COF nanoparticles to insulin is 1:2 to 2:1.

14. The method according to claim 10, wherein impregnating comprises forming a reaction mixture comprising the COF nanoparticles and insulin at room temperature, wherein the COF nanoparticles are impregnated with one or more insulin molecules.

15. The method according to claim 10, further comprising purifying the impregnated COF nanoparticles.

16. The method according to claim 15, wherein the purifying comprises centrifugation and washing with water.

17. The method according to claim 10, wherein the COF nanoparticles have a longest linear dimension of 75-300 nm.

18. The method according to claim 10, wherein the COF nanoparticles have 10-25 COF.

19. The method according to claim 10, wherein the molar ratio of DFP to TTA is 5:1.

20. The method according to claim 10, wherein the solvent is 1,4-dioxane.

21. The method according to claim 10, wherein the acid is acetic acid.

22. A method for treating an individual having or suspected of having diabetes, comprising orally administering a composition of claim 9, wherein the individual's glucose levels are normalized.

23. The method according to claim 22, wherein the individual has diabetes type 1 or diabetes type 2.

24. The method according to claim 23, wherein the individual has diabetes type 1.

25. A kit comprising COF nanoparticles according to claim 1 or the components to prepare a composition comprising COF nanoparticles of claim 1 or a composition comprising COF nanoparticles of claim 1.
